(19) United States
(12) Patent Application Publication
(10) Pub. No.: US 2025/0306015 A1
(43) Pub. Date: Oct. 2, 2025

(54) SINGLE MOLECULE-RESOLVED CHARACTERIZATION OF AFFINITY REAGENT KINETICS AND THERMODYNAMICS

(71) Applicant: Nautilus Subsidiary, Inc., Seattle, WA (US)

(72) Inventors: Vivekananda BUDAMAGUNTA, San Carlos, CA (US); Maria VILLANCIO-WOLTER, Foster City, CA (US); Filip BARTNICKI, Redwood City, CA (US); Maureen NEWMAN, Redwood City, CA (US); Devin SULLIVAN, San Diego, CA (US); Kara JUNEAU, San Carlos, CA (US); Parag MALLICK, San Mateo, CA (US); Sujal M. PATEL, Seattle, WA (US); Robert GROTHE, San Jose, CA (US)

(21) Appl. No.: 19/093,684

(22) Filed: Mar. 28, 2025

Related U.S. Application Data
(60) Provisional application No. 63/572,860, filed on Apr. 1, 2024.

(51) Int. Cl. G01N 33/543; G01N 33/68

(52) U.S. Cl. CPC G01N 33/54306; G01N 33/6803; G01N 2500/20

(57) ABSTRACT

The present disclosure provides methods of determining association rates or dissociation rates between affinity reagents and proteins. The methods can be configured to monitor a large number of proteins in parallel, for example, using arrays of proteins that are contacted with solutions containing affinity reagents. The methods can be further configured to detect the arrayed proteins at single-molecule resolution. Accordingly the methods allow a large population of proteins to be monitored on an individual basis. As such binding kinetics and thermodynamics can be determined on a population level while allowing individual interactions to be evaluated.

### CROSS REFERENCE

This application claims priority to U.S. Provisional Application No. 63/572,860, filed on Apr. 1, 2024, which is incorporated herein in its entirety by reference.

### BACKGROUND

Selection methods for the generation of affinity reagents are typically designed to select for binding reagents with high affinity and specificity for a single epitope or protein. For some applications it may be useful to select binding reagents which bind multiple epitopes, or to characterize the binding patterns of binding reagents which are not specific for a single protein or epitope. These promiscuous affinity reagents can provide advantages for combinatorial methods of identifying a large variety of different analytes, such as proteins, using a relatively small variety of affinity reagents. See, for example, U.S. Pat. No. 10,473,654 US Pat. App. Pub. Nos. 2020/0318101 A1 or 2023/0114905 A1 or Egertson et al., BioRxiv (2021), DOI: 10.1101/2021.10.11.463967, each of which is incorporated herein by reference. Furthermore, some research or clinical applications benefit from affinity reagents that demonstrate high avidity, increased avidity being correlated with reduced dissociation rate. The present disclosure provides methods, systems and compositions that can be configured to provide efficient selection or characterization of affinity reagents having desired binding properties such as a desired association rate, or dissociation rate. The rates can be helpful for identifying affinity reagents having high avidity or promiscuity.

### SUMMARY

The present disclosure provides a method of characterizing affinity reagents. The method can include steps of: (a) contacting a plurality of affinity reagents to a plurality of binding targets, (b) detecting at single-analyte resolution a first quantity of affinity reagents bound to binding targets of the plurality of binding targets at a first timepoint, (c) detecting at single-analyte resolution a second quantity of affinity reagents bound to binding targets of the plurality of binding targets at a second timepoint, and (d) based upon a difference between the first quantity and second quantity of affinity reagents bound to binding targets of the plurality of binding targets, determining an association rate of the affinity reagents for the binding targets.

In another aspect, provided herein is a method of characterizing an affinity reagent that can comprise the steps of: (a) contacting a plurality of affinity reagents to a plurality of binding targets, (b) detecting at single-analyte resolution a first quantity of affinity reagents bound to binding targets of the plurality of binding targets at a first timepoint, (c) detecting at single-analyte resolution a second quantity of affinity reagents bound to binding targets of the plurality of binding targets at a second timepoint, and (d) based upon a difference between the first quantity and second quantity of affinity reagents bound to binding targets of the plurality of binding targets, determining a dissociation rate of the affinity reagents for the binding targets.

In another aspect, provided herein is a system for characterizing affinity reagents, which can comprise: (a) a solid support comprising a plurality of binding targets, wherein the solid support comprises a plurality of addresses, wherein only one binding target of the plurality of binding targets is immobilized to each address of the plurality of addresses, and wherein each address is individually resolvable from each other address of the plurality of addresses, (b) a fluid comprising a plurality of affinity reagents, wherein each affinity reagent comprises a detectable label that is configured to produce optical signals, (c) a fluidics system that is configured to deliver the fluid comprising the plurality of affinity reagents to the solid support, (d) an optical detector, wherein the optical detector is configured to detect optical signals from detectable labels of affinity reagents at addresses of the plurality of addresses, and (e) a processor, wherein the processor is configured to receive data comprising presence or absence of an optical signal at each address of the plurality of addresses at a first timepoint and a second timepoint, and wherein the processor is further configured to determine an association rate of the affinity reagents for the binding targets based upon the received data for the first timepoint and the second timepoint.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF DRAWINGS

FIG. 1 shows a plot of HSP Lobe concentration vs. the percent of array addresses where Lobe was detected to be colocalized with a peptide at an address of the array.

FIG. 2 shows a plot of time vs. the percent of array addresses where Lobe was detected to be colocalized with a peptide at an address of the array.

FIG. 3A shows a photobleaching analysis of fluorescently labelled affinity reagents bound to peptides, wherein the peptides have a trimeric amino acid sequence epitope (DTR) that is recognized by the affinity reagents. FIG. 3B shows a photobleaching analysis of fluorescently labelled affinity reagents bound to peptides, wherein the peptides have a trimeric amino acid sequence epitope (WNK) that is recognized by the affinity reagents. FIG. 3C shows a photobleaching analysis of fluorescently labelled affinity reagents bound to peptides, wherein the peptides have a trimeric amino acid sequence epitope (YWL) that is recognized by the affinity reagents.

FIG. 4A shows a photobleaching analysis of fluorescently labelled affinity reagents bound to peptides, wherein the peptides have a trimeric amino acid sequence epitope (DTR) that is recognized by the affinity reagents, and wherein the conditions are the same as those used for FIG. 3A except 10 mM ascorbate is present. FIG. 4B shows a photobleaching analysis of fluorescently labelled affinity reagents bound to peptides, wherein the peptides have a trimeric amino acid sequence epitope (WNK) that is recognized by the affinity reagents, and wherein the conditions are the same as those used for FIG. 3A except 10 mM ascorbate is present. FIG. 4C shows a photobleaching analysis of fluorescently labelled affinity reagents bound to peptides, wherein the peptides have a trimeric amino acid sequence epitope (YWL) that is recognized by the affinity reagents, and wherein the conditions are the same as those used for FIG. 3A except 10 mM ascorbate is present.

FIG. 5 shows a plot of percent colocalization of Lobes with array addresses over 35 minutes and under conditions for dissociation of the Lobes from the addresses.

FIG. 6 shows a plot of percent colocalization of Lobes with array addresses over 220 minutes, wherein the data was acquired under conditions for detecting association kinetics from 0 to 150 minutes and the data was acquired under conditions for detecting dissociation kinetics from 151 to 230 minutes.

FIG. 7 shows a diagrammatic representation of a method for determining association rates for binding of affinity reagents to proteins at addresses of an array.

FIG. 8 shows a diagrammatic representation of a method for determining dissociation rates for binding of affinity reagents to proteins at addresses of an array.

FIG. 9 shows a diagrammatic representation of a method for determining dissociation rates for and identifying affinity reagents that interact with proteins at addresses of an array.

FIG. 10 depicts a diagram of a system that is configured to perform certain methods set forth herein, in accordance with some embodiments.

FIG. 11A illustrates a first configuration of affinity reagents bound to analytes under an equilibrium condition. FIG. 11B illustrates a second configuration of affinity reagents bound to analytes under the equilibrium condition.

### DETAILED DESCRIPTION

The present disclosure provides molecular assays for characterizing kinetics of association (i.e. binding) and dissociation for binding partners. The assays are particularly useful for characterizing interactions between affinity reagents and proteins and the assays will be exemplified herein in the context of these binding partners. However, the assays can be extended to any of a variety of analytes that bind to affinity reagents.

The assays set forth herein are particularly well suited for monitoring interactions between a plurality of immobilized proteins and solution phase affinity reagents. The assays can be configured for single molecule resolution, for example, detecting association between proteins distributed to addresses of an array, whereby each protein is spatially separated from all other proteins in the array. The spatial separation allows detection of binding between each protein and a respective affinity reagent to be resolved. The array format allows interactions at multiple array addresses to be detected and evaluated in parallel. As such, the format provided a multiplexed, single molecule-resolved binding assay.

Benefits of assays performed in a multiplexed, single molecule-resolved configuration include the ability to characterize a large number of binding events in parallel, thereby providing statistical rigor to analysis of results. Moreover, because binding events are individually resolved, subpopulations of proteins that have differing binding behavior can be identified. The observation of these differences can be indicative of differences in the structure, conformation or post-translational modification state for the subpopulations. This information can in turn be valuable for identifying biological phenotypes in research or clinical settings.

The assays set forth herein are useful for screening or profiling affinity reagents. For example, multiplexed, single molecule-resolved configurations provide a characterization that is indicative of how uniformly an affinity reagent interacts with a large and uniform population of proteins. In some cases, it may be desirable to select an affinity reagent that shows little to no variance when interacting with a particular protein species. In other cases, an affinity reagent that is sensitive to differences in structure, conformation or post-translational modification state of the protein species may be desired. More generally, multiplexed, single molecule-resolved configurations can be useful for determining failure modes of a particular affinity reagent species. These results can inform efforts to improve or modify affinity reagents for an intended use. Similarly, the results can be used to guide efforts to identify desired conditions for subsequent binding. For example, conditions can be varied in an assay set forth herein and the results can be used to identify conditions that are suited for a downstream assay.

Terms used herein will be understood to take on their ordinary meaning in the relevant art unless specified otherwise. Several terms used herein and their meanings are set forth below.

As used herein, the term “address” refers to a location in an array where a particular analyte (e.g. protein) is present. An address can contain a single analyte or, alternatively, it can contain a population of several analytes. Optionally, a population of analytes at an address can be identical (i.e. an ensemble of the analytes). Alternatively, an address can include a population of different analytes. Addresses are typically discrete. The discrete addresses can be contiguous, or they can be separated by interstitial spaces. An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

As used herein, the term “affinity reagent” refers to a molecule or other substance that is capable of specifically or reproducibly binding to an analyte (e.g. protein). An affinity reagent can be larger than, smaller than or the same size as the analyte. An affinity reagent may form a reversible or irreversible bond with an analyte. An affinity reagent may bind with an analyte in a covalent or non-covalent manner. Affinity reagents may include reactive affinity reagents, catalytic affinity reagents (e.g., kinases, proteases, etc.) or non-reactive affinity reagents (e.g., antibodies or fragments thereof). An affinity reagent can be non-reactive and non-catalytic, thereby not permanently altering the chemical structure of an analyte to which it binds. Affinity reagents that can be particularly useful for binding to proteins include, but are not limited to, antibodies or functional fragments thereof (e.g., Fab′ fragments, F(ab′)₂ fragments, single-chain variable fragments (scFv), di-scFv, tri-scFv, or microantibodies), affibodies, affilins, affimers, affitins, alphabodies, anticalins, avimers, DARPins, monobodies, nanoCLAMPs, nucleic acid aptamers, protein aptamers, lectins or functional fragments thereof. Affinity reagents can include pharmaceutical molecules, toxin molecules, or metabolites. The terms “affinity agent” and “affinity reagent” are used synonymously herein. Two affinity reagent molecules are considered to be identical species when the molecules have the same chemical structure and/or the same binding affinity for a given epitope.

As used herein, the term “antibody” refers to a protein that binds to an antigen or epitope via at least one complementarity determining region (CDR). An antibody can include all elements of a full-length antibody. However, an antibody need not be full length and functional fragments can be particularly useful for many uses. The term “antibody” as used herein encompasses full length antibodies and functional fragments thereof.

As used herein, the term “array” refers to a population of analytes (e.g. proteins) that are associated with unique identifiers such that the analytes can be distinguished from each other. A unique identifier can be, for example, a solid support (e.g. particle or bead), address on a solid support, tag, label (e.g. luminophore), or barcode (e.g. nucleic acid barcode) that is associated with an analyte and that is distinct from other identifiers in the array. Analytes can be associated with unique identifiers by attachment, for example, via covalent bonds or non-covalent bonds (e.g. ionic bond, hydrogen bond, van der Waals forces, electrostatics etc.). An array can include different analytes that are each attached to a particular unique identifier. An array can include different unique identifiers that are attached to the same or similar species of analyte. An array can include separate solid supports or separate addresses that each bear a different analyte, wherein the different analytes can be identified according to the locations of the solid supports or addresses.

As used herein, the term “attached” refers to the state of two things being joined, fastened, adhered, connected or bound to each other. Attachment can be covalent or non-covalent. For example, a particle can be attached to a protein by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions. A covalent attachment between moieties A and B includes an uninterrupted chain of covalent bonds between moieties A and B, whereas a non-covalent attachment between moieties A and B include at least one non-covalent bond in a chain of bonds between moieties A and B.

The term “comprising” is intended herein to be open-ended, including not only the recited elements, but further encompassing any additional elements.

As used herein, the term “covalent,” when used in reference to a bond between atoms or moieties of a molecule, refers to bonding due to sharing of a pair of electrons between the two atoms or moieties. Covalent interactions can include reversible and irreversible binding interactions. Covalent interaction can arise due to a chemical reaction between a first reactive moiety and a second reactive moiety, optionally in the presence of a third intermediary or catalytic moiety. Covalent binding interactions can form between two atoms or moieties due to various chemical mechanisms, including addition, substitution, elimination, oxidation, and reduction. In some cases, a covalent binding interaction may be formed by a Click-type reaction, as set forth herein (e.g., methyltetrazine (mTz)-tetracyclooctylene (TCO), azide-dibenzocyclooctene (DBCO), thiol-epoxy). In some cases, a ligand-receptor-type binding interaction can also form a covalent binding interaction. For example, SpyCatcher-SpyTag, SnoopCatcher-SnoopTag, and SdyCatcher-SdyTag are receptor-ligand binding pairs that can form covalent binding interactions due to isopeptide bond formation. Additional useful covalent interactions can include coordination bond formation, such as between a metal-containing substrate and a ligand. Exemplary coordination bonds can include silicon-silane, metal oxide-phosphate, and metal oxide-phosphonate. Useful reagents and mechanisms for forming covalent binding interactions, including bioorthogonal binding interactions, as set forth herein, are provided in U.S. Pat. Nos. 11,203,612 and 11,505,796, each of which is herein incorporated by reference in its entirety. A linker having a chain of multiple bonds that connects two substances is considered to be a covalent linker if all of the bonds in the chain that connects the two substances are covalent. A linker is covalent if the substances that it connects can not be separated by breaking a non-covalent bond.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “epitope” refers to a molecule or part of a molecule, which is recognized by or binds specifically to an affinity reagent or paratope. Epitopes may include amino acid sequences that are sequentially adjacent in the primary structure of a protein, or amino acids that are structurally adjacent in the secondary, tertiary or quaternary structure of a protein. An epitope can be, or can include, a moiety of a protein that arises due to a post-translational modification, such as a phosphate (e.g. phosphotyrosine, phosphoserine, phosphothreonine, or phosphohistidine). An epitope can optionally be recognized by or bound to an antibody. However, an epitope need not necessarily be recognized by any antibody, for example, instead being recognized by an aptamer, mini-protein or other affinity reagent. An epitope can optionally bind an antibody to elicit an immune response. However, an epitope need not necessarily participate in, nor be capable of, eliciting an immune response.

As used herein, the term “fluid-phase,” when used in reference to a molecule or particle, means the molecule or particle is in a state wherein it is mobile in a fluid, for example, being capable of diffusing through the fluid.

As used herein, the terms “group” and “moiety” are intended to be synonymous when used in reference to the structure of a molecule. The terms refer to a component or part of the molecule. The terms do not necessarily denote the relative size of the component or part compared to the rest of the molecule, unless indicated otherwise.

As used herein, the term “immobilized,” when used in reference to a molecule or particle that is in contact with a fluid phase, refers to the molecule or particle being prevented from diffusing in the fluid phase. For example, immobilization can occur due to the molecule being confined at, or attached to, a solid phase substance. Immobilization can be temporary (e.g. for the duration of one or more steps of a method set forth herein) or permanent. Immobilization can be reversible or irreversible under conditions utilized for a method, system or composition set forth herein.

As used herein, the term “label” refers to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like. A label that produces an optical signal can be referred to as an “optical label.” A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint.

As used herein, the term “non-covalent,” when used in reference to a bond between atoms or moieties of a molecule, refers to bonding due a mechanism other than electron pair-sharing between the two atoms or moieties. Non-covalent interaction can arise due to an electrostatic or magnetic interaction between moieties and/or atoms. Non-covalent binding interactions can include electrostatic interactions such as ionic bonding, hydrogen bonding, halogen bonding, Van der Waals interactions, Pi-Pi stacking, Pi-ion interactions, Pi-polar interactions, or magnetic interactions. In some cases, a non-covalent interaction may include hybridization of a first oligonucleotide to a complementary second oligonucleotide. In some cases, a non-covalent interaction may form between a receptor and ligand, such as streptavidin-biotin. Other useful non-covalent interactions can include affinity reagent-target interactions, such as antibody-epitope or aptamer-epitope interactions. A linker having a chain of multiple bonds that connects two substances is considered to be a non-covalent linker if at least one of the bonds in the chain that connects the two substances is non-covalent. A linker is non-covalent if the substances that it connects can be separated by breaking a non-covalent bond.

As used herein, the term “nucleic acid origami” refers to a nucleic acid construct having an engineered tertiary or quaternary structure. A nucleic acid origami may include DNA, RNA, PNA, modified or non-natural nucleic acids, or combinations thereof. A nucleic acid origami may include a plurality of oligonucleotides that hybridize via sequence complementarity to produce the engineered structuring of the origami. A nucleic acid origami may include sections of single-stranded or double-stranded nucleic acid, or combinations thereof. Exemplary nucleic acid origami structures may include nanotubes, nanowires, cages, tiles, nanospheres, blocks, and combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

As used herein, the term “paratope” refers to a molecule or portion thereof, which recognizes or binds specifically to an epitope. A paratope may include an antigen binding site of an antibody. A paratope may include at least 1, 2, 3, or more complementarity-determining regions of an antibody. A paratope need not necessarily be present in nor derived from an antibody, for example, instead being present in a nucleic acid aptamer, lectin, streptavidin, miniprotein or other affinity reagent. A paratope need not necessarily participate in, nor be capable of, eliciting an immune response.

As used herein, the term “pitch” refers to the distance between corresponding points on two nearest neighbor addresses in an array. For example, the corresponding points can be the centers of two adjacent addresses (e.g., center to center distance). The pitch for adjacent addresses can be greater than, or equal to, the diameter or maximum length of the addresses. The pitch for addresses of an array can be at least 10 nm, 25 nm, 100 nm, 250 nm, 500 nm, 1 micron, 5 microns or greater. Alternatively or additionally, the pitch for addresses of an array can be at most 5 microns, 1 micron, 500 nm, 250 nm, 100 nm, 25 nm, 10 nm or less. In some cases, for example in cases of an array having a uniform pattern or repeating pattern of addresses an array can be described in terms of average pitch. The average pitch for an array can be at least 10 nm, 25 nm, 100 nm, 250 nm, 500 nm, 1 micron, 5 microns or greater. Alternatively or additionally, the average pitch for an array can be at most 5 microns, 1 micron, 500 nm, 250 nm, 100 nm, 25 nm, 10 nm or less.

As used herein, the term “post-translational modification” refers to a change to the chemical composition of a protein compared to the chemical composition encoded by the gene for the protein. Exemplary changes include those that alter the presence, absence or relative arrangement of different regions of amino acid sequence (e.g., splicing variants, or protein processing variants of a single gene), or due to presence or absence of different moieties on particular amino acids (e.g., post-translationally modified variants of a single gene). A post-translational modification can be derived from an in vivo process or in vitro process. A post-translational modification can be derived from a natural process or a synthetic process. Exemplary post-translational modifications include those classified by the PSI-MOD ontology. See Smith, L. M. et al. Nat. Methods, 2013, 10, 186-187.

As used herein, the term “protein” refers to a molecule comprising two or more amino acids joined by a peptide bond. A protein may also be referred to as a polypeptide, oligopeptide or peptide. Although the terms “protein,” “polypeptide,” “oligopeptide” and “peptide” may optionally be used to refer to molecules having different characteristics, such as amino acid composition, amino acid sequence, amino acid length, molecular weight, origin of the molecule or the like, the terms are not intended to inherently include such distinctions in all contexts. A protein can be a naturally-occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

As used herein, the term “retaining component” refers to a particle, molecule or material to which one or more moieties of an affinity reagent are attached. Exemplary retaining components include, but are not limited to, structured nucleic acid particles, nucleic acid origami, particles made of solid support materials, or polymers such as branched polymers or dendrimers. Affinity reagent moieties that can be attached to a retaining component, directly or indirectly, include for example, one or more paratopes, one or more labels, one or more antibodies, one or more nucleic acid aptamers, one or more nucleic acid tags or the like.

As used herein, the term “single,” when used in reference to an object such as an analyte, means that the object is individually manipulated or distinguished from other objects. A single analyte can be a single molecule (e.g. single protein), a single complex of two or more molecules (e.g. a multimeric protein having two or more separable subunits, a single protein attached to a structured nucleic acid particle or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

As used herein, the term “single-analyte resolution” refers to the detection of, or ability to detect, an analyte on an individual basis, for example, as distinguished from its nearest neighbor in an array. The term when used in reference to a single-analyte array, refers to detection of a single-analyte under the conditions that: 1) the single-analyte is detected by a signal with a magnitude that exceeds the magnitude of background signals for the detection system, and 2) the single-analyte is detected by a signal at a location that is spatially separated from the location of a signal corresponding to a different single-analyte. In some cases, a signal corresponding to a first single-analyte may be considered spatially resolved from a signal corresponding to a second single-analyte if a signal minimum occurs between the locations of the two single-analytes with a magnitude that is substantially less than an average or peak signal maximum of one or both signal maxima corresponding to the first and second single analytes. For example, a signal minimum between two signal maxima corresponding respectively to a first single analyte and a second single analyte may have a magnitude that is no more than about 49% 40%, 30%, 20%, 10%, 5%, 1%, or less than 1% of an average or peak signal maximum of the two signal maxima. In some cases, signals corresponding to two or more analytes may be considered spatially resolved if a spatial resolution criterion is achieved, such as the Rayleigh Criterion.

As used herein, the term “solid support” refers to a substrate that is insoluble in aqueous liquid. Optionally, the substrate can be rigid. The substrate can be non-porous or porous. The substrate can optionally be capable of taking up a liquid (e.g. due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In particular configurations, a flow cell contains the solid support such that fluids introduced to the flow cell can interact with a surface of the solid support to which one or more components of a binding event (or other reaction) is attached.

As used herein, the term “structured nucleic acid particle” or “SNAP” refers to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same sequence length as the SNAP. The compacted three-dimensional structure can optionally be characterized with regard to tertiary structure. For example, a SNAP can be configured to have an increased number of internal binding interactions between regions of a polynucleotide strand, less distance between the regions, increased number of bends in the strand, and/or more acute bends in the strand, as compared to a nucleic acid molecule of similar length in a random coil or other non-structured state. Alternatively or additionally, the compacted three-dimensional structure can optionally be characterized with regard to tertiary or quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure of a SNAP can be configured to be more dense than a nucleic acid molecule of similar length in a random coil or other non-structured state. A SNAP may contain DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as proteins, reactive moieties, or detectable labels) or are configured to be attached to other molecules (e.g., by functional groups). A SNAP may include engineered or rationally designed structures. Exemplary SNAPs include nucleic acid origami and nucleic acid nanoballs.

As used herein, the term “unique identifier” refers to a moiety, object or substance that is associated with an analyte and that is distinct from other identifiers, throughout one or more steps of a process. The moiety, object or substance can be, for example, a solid support such as a particle or bead; a location on a solid support; an address in an array; a tag; a label such as a luminophore; a molecular barcode such as a nucleic acid having a unique nucleotide sequence or a protein having a unique amino acid sequence; or an encoded device such as a radiofrequency identification (RFID) chip, electronically encoded device, magnetically encoded device or optically encoded device. The process in which a unique identifier is used can be an analytical process, such as a method for detecting, identifying, characterizing or quantifying an analyte; a separation process in which at least on analyte is separated from other analytes; or a synthetic process in which an analyte is modified or produced. The unique identifier can be associated with an analyte via immobilization. For example, a unique identifier can be covalently or non-covalently (e.g. ionic bond, hydrogen bond, van der Waals forces etc.) attached to an analyte. A unique identifier can be exogenous to an associated analyte, for example, being synthetically attached to the associated analyte. Alternatively, a unique identifier can be endogenous to the analyte, for example, being attached or associated with the analyte in the native milieu of the analyte.

The embodiments set forth below and recited in the claims can be understood in view of the above definitions.

The present disclosure provides methods, compositions, and systems for characterizing affinity reagents. The methods provided in the present disclosure may be useful for determining association rates and/or dissociation rates between affinity reagents and binding targets, such as proteins or other analytes. The methods of the present disclosure may include the steps of: (a) contacting a plurality of affinity reagents to a plurality of binding targets, (b) detecting at single-analyte resolution a first quantity of affinity reagents bound to binding targets of the plurality of binding targets for a first timepoint, (c) detecting at single-analyte resolution a second quantity of affinity reagents bound to binding targets of the plurality of binding targets for a second timepoint, and (d) based upon a difference between the first quantity and second quantity of affinity reagents bound to binding targets of the plurality of binding targets, determining an association rate or dissociation rate of the affinity reagents for the binding targets.

One aspect of measuring binding kinetics and/or equilibrium with single-analyte resolution is the ability to observe the binding interactions of an affinity reagent to each individual binding target. For example, during association of affinity reagents to binding targets, some quantity of affinity reagents may dissociate from binding targets between a first timepoint and a second timepoint, but a greater quantity of binding targets may become bound by an affinity reagent than the quantity of affinity reagents that dissociated. Likewise, during dissociation, a greater quantity of affinity reagents may dissociate than a quantity of affinity reagents that associate to binding targets. Further, at equilibrium, different sets of binding targets can be detected as bound by an affinity reagent between two timepoints, but the quantity of bound binding targets in each set should be equal.

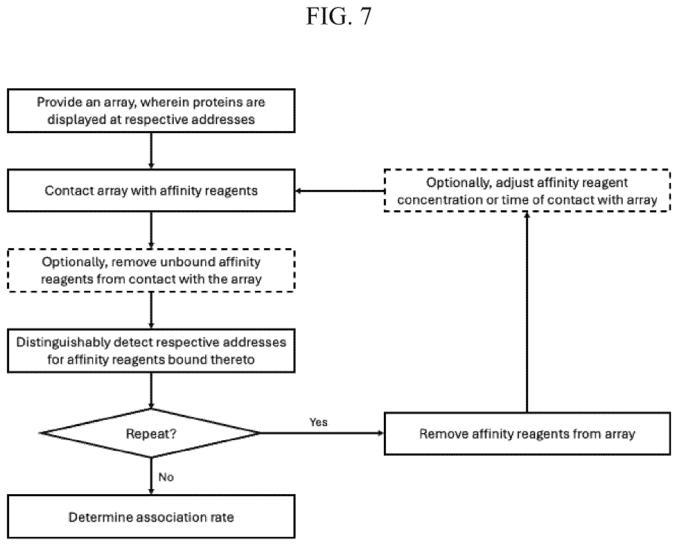

In some configurations, the methods can be configured to determine association rates between proteins and affinity reagents. Accordingly, an affinity reagent characterization method can be configured to include steps of: (a) providing an array, wherein the array includes a plurality of addresses, wherein a plurality of proteins is attached to the plurality of addresses, and wherein individual addresses of the array are each attached to a single protein of the plurality of proteins; (b) performing an assay, including (i) contacting the array with a set of affinity reagents, wherein the affinity reagents have optical labels, (ii) detecting binding of the affinity reagents to proteins at addresses of the array, wherein the detecting includes acquiring optical signals from the optical labels at respective addresses of the array, wherein the respective addresses are individually resolved, and (iii) removing affinity reagents from the array, wherein steps (i) through (iii) are repeated for a plurality of cycles, each of the cycles using another set of affinity reagents instead of the set of affinity reagents, wherein affinity reagent species composition of the set of affinity reagents is identical to the other set of affinity reagents; and (c) determining an association rate between the affinity reagents and the proteins based on the assay. A diagrammatic representation of the method is shown in FIG. 7.

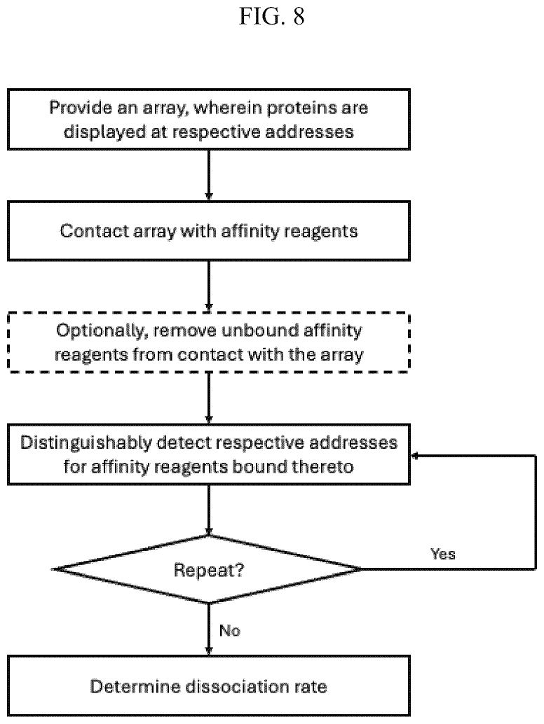

In particular configurations, a method of the present disclosure can be configured to determine dissociation rates between proteins and affinity reagents. Accordingly, an affinity reagent characterization method can be configured to include steps of: (a) providing an array, wherein the array includes a plurality of addresses, wherein a plurality of proteins is attached to the plurality of addresses, and wherein individual addresses of the array are each attached to a single protein of the plurality of proteins; (b) performing an assay, including (i) contacting the array with a set of affinity reagents, wherein the affinity reagents include optical labels, and wherein the affinity reagents bind to proteins at addresses of the array, (ii) detecting proteins at addresses of the array that are bound to affinity reagents of the set, wherein the detecting includes acquiring optical signals from the optical labels at respective addresses of the array, wherein the respective addresses are individually resolved, and (iii) repeating step (ii) for a plurality of cycles, thereby detecting a decay in optical signals at the respective addresses of the array; and (c) determining a dissociation rate between the affinity reagents and the proteins based on the assay. A diagrammatic representation of the method is shown in FIG. 8.

In yet other configurations, a method of the present disclosure can be used to determine association rates and dissociation rates between proteins and affinity reagents. For example, an affinity reagent characterization method can include steps of: (a) providing an array, wherein the array includes a plurality of addresses, wherein a plurality of proteins is attached to the plurality of addresses, and wherein individual addresses of the array are each attached to a single protein of the plurality of proteins; (b) performing an assay, including (i) contacting the array with a set of affinity reagents, wherein the affinity reagents have optical labels, (ii) detecting binding of the affinity reagents to proteins at addresses of the array, wherein the detecting includes acquiring optical signals from the optical labels at respective addresses of the array, wherein the respective addresses are individually resolved, (iii) removing affinity reagents from the array, wherein steps (i) through (iii) are repeated for a plurality of cycles, each of the cycles using another set of affinity reagents instead of the set of affinity reagents, wherein affinity reagent species composition of the set of affinity reagents is identical to the other set of affinity reagents, (iv) contacting the array with a further set of affinity reagents, wherein affinity reagent species composition of the set of affinity reagents is identical to the further set of affinity reagents, and wherein affinity reagents of the further set include optical labels, (v) detecting proteins at addresses of the array that are bound to affinity reagents of the further set, wherein the detecting includes acquiring optical signals from the optical labels at respective addresses of the array, wherein the respective addresses are individually resolved, and (vi) repeating step (v) for a plurality of cycles, thereby detecting a decay in optical signals at the respective addresses of the array; (c) determining an association rate between the affinity reagents and the proteins based on the assay; and (d) determining a dissociation rate between the affinity reagents of the further set and the proteins based on the assay.

A method set forth herein can be carried out on a solid support. One or more proteins can be attached to a solid support and contacted with a fluid containing one or more affinity reagents. A solid support can be composed of a substrate that is insoluble in aqueous liquid. The substrate can have any of a variety of other characteristics such as being rigid, non-porous or porous. Exemplary solid supports include, but are not limited to, glass and modified or functionalized glass, plastics (including acrylics, polystyrene or copolymers of styrene and other materials, polypropylene, polyethylene, polybutylene, polyurethanes, Teflon™, cyclic olefins, or polyimides etc.), nylon, ceramics, resins, Zeonor™, silica or silica-based materials including silicon and modified silicon, carbon, metals, inorganic glasses, optical fiber bundles, gels, and polymers. In some cases, a solid support may include silicon, fused silica, quartz, mica, or borosilicate glass. In particular configurations an array of proteins or other analytes can immobilized on a solid support and fluids can be introduced to the flow cell thereby allowing components in the fluid, such as affinity reagents, to interact with the proteins or other analytes on a surface of the solid support.

Methods of the present disclosure may include measuring temporal aspects of binding interactions between analytes and affinity reagents. A method may include a step of providing an array of analytes, e.g., a single-analyte array of analytes. Useful analytes that may be provided on an array are not particularly limited, and can include polypeptides, polynucleotides, polysaccharides, lipids, metabolites, toxins, small molecule compounds (e.g., molecules of no more than 1 kiloDalton), pharmaceutical small molecule compounds, macromolecules (e.g., molecules of greater than 1 kiloDalton), pharmaceutical macromolecular compounds (e.g., monoclonal or polyclonal antibodies, etc.), synthetic or natural polymer particles, synthetic organic particles (e.g., carbon nanoparticles, carbon nanotubes, etc.), synthetic inorganic particles (e.g., metal, metal oxide, metal nitride, metal carbide, semiconductor, ceramic, or mineral nanoparticles, or combinations thereof), and combinations thereof.

A method of the present disclosure can be carried out at single analyte resolution. As such, a single analyte, such as a single protein (i.e. one and only one protein), can be individually manipulated or distinguished using a method set forth herein. A single protein may be resolved based on, for example, spatial or temporal separation from other proteins. Reference herein to a ‘single protein’ in the context of a composition, apparatus or method set forth herein does not necessarily exclude application of the composition, apparatus or method to multiple single proteins that are manipulated or distinguished individually, unless indicated to the contrary. For example, a single protein assay can resolve proteins individually while also being multiplexed to allow a plurality of individually resolved proteins to be detected in parallel.

Alternatively to single-analyte resolution, a method can be carried out at ensemble resolution or bulk resolution. Bulk resolution configurations acquire a composite signal from a plurality of analytes that are not resolved from each other, such as a plurality of proteins attached to an address of an array. Ensemble resolution configurations acquire a composite signal from a first collection of proteins or affinity reagents in a sample, such that the composite signal is distinguishable from signals generated by a second collection of proteins or affinity reagents in the sample. For example, the ensembles can be located at respective addresses in an array. Accordingly, the composite signal obtained from each address will be an average of signals from the ensemble, yet signals from different addresses can be distinguished from each other. An ensemble resolution protein assay can be multiplexed to allow each protein ensemble in a plurality of proteins ensembles to be resolved from other protein ensembles in the plurality of ensembles, while also allowing a plurality of ensembles to be detected in parallel.

A composition, apparatus or method set forth herein can be configured to contact one or more proteins with one or more affinity reagents. For example, an array can include a plurality of addresses, wherein a plurality of proteins is attached to the plurality of addresses, wherein individual addresses of the array are each attached to a single protein of the plurality of proteins and wherein individual proteins of the plurality of proteins are each attached to a single address of the array. Some or all addresses in an array can be attached to identical species of protein. For example, the identical species of protein can be products of the same gene or can have identical amino acid sequences. Identical species of proteins can further have identical post-translational modifications. However, in some cases the assay configuration used is not adequately sensitive to distinguish differences in the number or type of post-translational modifications present in two or more proteins, in which case the proteins can be considered as apparently identical species. For example, a first address in an array can be attached to a protein having a given amino acid sequence and a post-translational modification at a particular position in the amino acid sequence, whereas a second address in the array can be attached to a protein having the given amino acid sequence but lacking the post-translational modification at the particular position. When using affinity reagents that are specific for one of the post-translational modifications in this example, the proteins can be considered as different species, but when using affinity reagents that do not distinguish one protein from the other, the proteins can be considered as apparently identical species.

In particular configurations, an array can include addresses that are attached to different species of protein, respectively. For example, an array can include a first address that is attached to a first protein and can also include a second address that is attached to a second protein, wherein the amino acid sequence of the first protein differs substantially from the amino acid sequence of the second protein. In this example, the first and second proteins can be encoded by different genes. It will be understood that an array can include a first subset of addresses that are each attached to proteins having a first amino acid sequence (or encoded by a first gene) and can also include a second subset of addresses that are each attached to proteins having a second amino acid sequence (or encoded by a second gene), wherein the first amino acid sequence differs substantially from the second amino acid sequence (or wherein the first gene is different from the second gene). Similarly, an array can include a first subset of addresses that are each attached to proteins that are encoded by a first gene and can also include a second subset of addresses that are each attached to proteins that are encoded by a second gene, wherein the first gene is different from the second gene.

In particular configurations, an array can include addresses that are attached to a plurality of different proteins (e.g., proteins having different primary amino acid sequences), in which proteins of the different proteins comprise an epitope Θ (e.g., Θ is an amino acid sequence of about 2, 3, 4, 5, 6, or 7 contiguous amino acids; Θ is an amino acid sequence comprising a post-translational modification). In particular configurations, an array can include addresses that are attached to a plurality of different proteins, in which each protein of a first set of proteins of the different proteins comprises an epitope Θ, and in which each protein of a second set of proteins of the different proteins does not comprise the epitope Θ.

Proteins can be attached to addresses of an array such that the proteins are spatially resolved from each other. An array can include at least about 100, 1×10³, 1×10⁴, 1×10⁵, 1×10⁶, 1×10⁹, 1×10¹², or more addresses. Some or all of the addresses can be attached to identical species of protein. For example, at least 100, 1×10³, 1×10⁴, 1×10⁵, 1×10⁶, 1×10⁹, 1×10¹² or more addresses of an array can be attached to identical species of protein.

Addresses are typically discrete in an array. Discrete addresses that neighbor each other can be contiguous, or they can be separated by interstitial spaces. A plurality of addresses in an array can have a minimum, maximum or average pitch of at least about 10 nm, 100 nm, 250 nm, 500 nm, 1 micron, 2 microns, 10 microns or more. Alternatively or additionally, a plurality of addresses in an array used in a method set forth herein can have a minimum, maximum or average pitch of at most 10 microns, 2 microns, 1 micron, 500 nm, 250 nm, 100 nm, 10 nm or less. The relative positions of addresses in an array can be described according to separation between addresses and/or area of the addresses. For example, an array useful herein can have addresses that are separated by an average, minimum or maximum distance of less than 10 microns, 2 microns, 1 micron, 500 nm, 250 nm, 100 nm, 10 nm or less. Alternatively or additionally, an array can have addresses that are separated by an average, minimum or maximum distance of at least 10 nm, 100 nm, 250 nm, 500 nm, 1 micron, 2 microns, 10 microns or more. The average, minimum or maximum area (e.g. footprint) for addresses in an array can be less than 1 square millimeter, 500 square microns, 100 square microns, 10 square microns, 1 square micron, 100 square nanometers or less. Alternatively or additionally, the average, minimum or maximum area (e.g. footprint) for addresses in an array can be greater than 100 square nanometers, 1 square micron, 10 square microns, 100 square microns, 500 square microns, 1 square millimeter or more.

A solid support or a surface thereof may be configured to display a protein or a plurality of proteins. A solid support may contain one or more addresses in formed or prepared surfaces. Multiple addresses can be configured to form a pattern. In some cases, a solid support may contain one or more patterned, formed, or prepared surfaces that contain a plurality of addresses, with each address configured to display one or more protein. In some configurations, a solid support or a surface thereof may be patterned or formed to produce a repeating pattern of addresses. The deposition of proteins on the repeating pattern of addresses may be controlled by interactions between the solid support and the proteins such as, for example, electrostatic interactions, magnetic interactions, hydrophobic interactions, hydrophilic interactions, covalent interactions, or non-covalent interactions. Accordingly, the coupling of a protein at each address of an array may produce an array of proteins whose average spacing is relatively uniform. An ordered or patterned array of addresses may be characterized as having a regular geometry, such as a rectangular, triangular, polygonal, or annular grid. In other configurations, a solid support or a surface thereof may have a random or non-repeating pattern of addresses.

Proteins can be in a native (i.e. correctly folded) state or in a denatured state (i.e. unfolded or misfolded) state during one or more steps of a method set forth herein. For example, proteins can be denatured when bound by an affinity reagent and/or when detected in a method set forth herein. Denaturation can provide an advantage of increasing access of an affinity reagent to an epitope that is typically inaccessible in the native state of the protein. In other cases, it may be desirable to have a protein in a native state when bound to an affinity reagent. For example, apparent accessibility, or apparent lack of accessibility, of a native protein to an affinity reagent can provide information regarding the conformation of the protein. A protein can be denatured using any of a variety of techniques known in the art including, but not limited to, exposure to non-physiological temperatures (e.g. greater than 40° C., 50° C., 60° C., 75° C., 90° C. or more), strong acid (e.g. pH less than 5.0), strong base (e.g. pH greater than 9.0), chaotropic agents (e.g. urea, guanidinium chloride, sodium dodecyl sulfate), or organic solvents (e.g. chloroform or ethanol). Denatured proteins will generally lack tertiary structure and quaternary structure, or at least have tertiary structure and quaternary structure that is non-native. Denatured proteins typically lack native function, for example, lacking ability to bind their natural ligands or lacking ability to catalyze their natural reactions.

A protein can be attached to an address in an array using any of a variety of means. The attachment can be covalent or non-covalent. Exemplary covalent attachments include chemical linkers such as those utilizing click chemistry or other linkages known in the art or described in U.S. Pat. Nos. 11,203,612 or 11,505,796 or US Pat. App. Pub. No 2023/0167488 A1, each of which is incorporated herein by reference. Non-covalent attachment can be mediated by receptor-ligand interactions (e.g. (strept) avidin-biotin, antibody-antigen, or complementary nucleic acid strands), for example, in which the receptor is attached to the address and the ligand is attached to the protein or vice versa.

In particular configurations, a protein is attached to a solid support (e.g. an address in an array) via a retaining component. A particularly useful retaining component is a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful SNAPs. A nucleic acid nanoball can include a concatemeric repeat of amplified nucleotide sequences. The concatemeric amplicons can include complements of a circular template amplified by rolling circle amplification. Exemplary nucleic acid nanoballs and methods for their manufacture are described, for example, in U.S. Pat. No. 8,445,194, which is incorporated herein by reference. A nucleic acid origami can include one or more nucleic acids folded into any of a variety of overall shapes such as a disk, tile, cylinder, cone, sphere, cuboid, tubule, pyramid, polyhedron, or combination thereof. Examples of structures formed with DNA origami are set forth in Zhao et al. Nano Lett. 11, 2997-3002 (2011); Rothemund Nature 440:297-302 (2006); Sigle et al, Nature Materials 20:1281-1289 (2021); or U.S. Pat. Nos. 8,501,923 or 9,340,416, each of which is incorporated herein by reference. Further examples of structured nucleic acid particles are set forth below or in U.S. Pat. Nos. 11,203,612 or 11,505,796; or US Pat. App. Pub. No. 2022/0162684 A1 or 2023/0167488 A1, each of which is incorporated herein by reference.

An array of binding targets may be provided to a method or system set forth herein. An array of binding targets may comprise a plurality of binding targets that are individually immobilized at discrete addresses of a solid support. Useful configurations of arrays of binding targets are described in U.S. Pat. Nos. 11,970,693 and 11,993,865, each of which is herein incorporated by reference in its entirety. Alternatively, a method or system set forth herein may utilize a plurality of binding targets that are provided in a fluid phase.

A plurality of binding targets may be provided to a method set forth herein, in which the plurality of binding targets is immobilized on a solid support. A solid support can comprise a plurality of sites, in which the plurality of binding targets is immobilized to the plurality of sites. In some configurations, only one binding target of the plurality of binding targets may be immobilized to a site of the plurality of sites. In other configurations, two or more binding targets of the plurality of binding targets can be immobilized to a site of the plurality of sites. Array compositions provided herein, such as array compositions formed by the deposition of binding targets utilizing retaining components may be advantageous for a method set forth herein. A retaining component may facilitate co-localization of a binding target with a binding target barcode moiety (e.g., the binding target and binding target barcode moiety are both attached to the retaining component). A plurality of binding targets can be attached to a plurality of retaining components, in which each site of the plurality of sites is attached to a retaining component of the plurality of retaining components. In some configurations, each retaining component can be attached to only one binding target. In other configurations, each retaining component can be attached to two or more binding targets.

In some configurations, a plurality of binding targets can be immobilized on a solid support, in which the solid support comprises a mobile solid support (e.g., a bead, a particle, a microparticle, a nanoparticle, etc.). A plurality of binding targets immobilized on a mobile solid support may be suspended, solvated, or otherwise mobile in a fluidic medium. In some configurations, a mobile solid support may comprise a magnetic particle, an electrically-charged particle, or a sedimenting particle. In some configurations, a binding target may be attached to a mobile solid support by a retaining component.

In some configurations, a plurality of binding targets may be substantially homogeneous with respect to the structure of binding targets of the plurality of binding targets. For example, a plurality of binding targets can comprise a plurality of binding targets, each binding target having an identical primary amino acid sequence. In another example, a plurality of binding targets can comprise a plurality of binding targets, each binding target containing an identical proteoform of a single protein. It may be useful to provide a plurality of binding targets that is substantially homogeneous with respect to the structure of binding targets of the plurality of binding targets when screening and selecting for a binding reagent that is specific to a single protein.

Alternatively, in some configurations, a plurality of binding targets may be heterogeneous with respect to the structure of binding targets of the plurality of binding targets. In some configurations, a plurality of binding targets may be provided with a plurality of unique binding targets, as determined by primary amino acid sequence, in which each binding target of the plurality of binding targets comprises an epitope Θ of length n (where n equals at least about 2, 3, 4, 5, 6, 7, 8, 9, 10, 15, 20, 25, 30 or more than 30 amino acids), in which the epitope Θ has a structure X₁X₂ . . . Xₙ, where each X can be independently selected from any naturally-occurring, non-natural, or modified amino acid (e.g., each peptide of a plurality of binding target peptides contains an epitope having the amino acid sequence DTR).

In particular configurations, a plurality of binding targets may be provided, in which the plurality of binding targets contains at least about 10, 20, 50, 100, 200, 400, 1000, 2000, 5000, 10000, 15000, 20000, 50000, 100000, 1000000, or more than 1000000 sequence contexts of an epitope Θ of length n (where n equals at least about 2, 3, 4, 5, 6, 7, 8, 9, 10, 15, 20, 25, 30 or more than 30 amino acids), in which the epitope Θ has a structure X₁X₂ . . . . Xₙ, where each X can be independently selected from any naturally-occurring, non-natural, or modified amino acid (e.g., each peptide of a plurality of binding target peptides contains an epitope having the amino acid sequence DTR). Each sequence context may comprise a structure αΘβ, in which α and β are flanking amino acid sequences of epitope Θ, in which α and β can independently comprise about 0, 1, 2, 3, or more than 3 amino acids, and in which α and β can contain any naturally-occurring, non-natural, or modified amino acid. For example, in a particular configuration, a plurality of binding targets may comprise at least 400 sequence contexts of epitope Θ, in which α and β are each a single amino acid, and in which every permutation of α and β for the 20 naturally-occurring amino acids is present in the at least 400 sequence contexts (e.g., α=A, β=A; α=A, β=C; α=A, β=D, etc.).

In some configurations, a plurality of binding targets may be provided with a plurality of unique binding targets, as determined by primary amino acid sequence, in which each binding target of the plurality of binding targets comprises an epitope Θ of length n (where n equals at least about 2, 3, 4, 5, 6, 7, 8, 9, 10, 15, 20, 25, 30, or more than 30 amino acids), in which the epitope Θ has a structure X₁X₂ . . . . Xₙ, where each X can be independently selected from any naturally-occurring, non-natural, or modified amino acid, and in which at least one residue X of epitope Θ contains a modified structure (e.g., a post-translational modification, a non-natural amino acid). For example, a plurality of binding targets can comprise a plurality of binding targets, in which each binding target of the plurality of binding targets comprises an epitope Θ, in which Θ has a structure DC*R, in which C* can be any post-translational modification of the amino acid cysteine.

In particular cases, a plurality of binding targets may be provided, in which the plurality of binding targets contains at least about 10, 20, 50, 100, 200, 400, 1000, 2000, 5000, 10000, 15000, 20000, 50000, 100000, 1000000, or more than 1000000 sequence contexts of an epitope Θ of length n (where n equals at least about 2, 3, 4, 5, 6, 7, 8, 9, 10, 15, 20, 25, 30, or more than 30 amino acids), in which the epitope Θ has a structure X₁X₂ . . . . Xₙ, where each X can be independently selected from any naturally-occurring, non-natural, or modified amino acid. Each sequence context may comprise a structure αΘβ, in which α and β are flanking amino acid sequences of epitope Θ, in which α and β can independently comprise about 0, 1, 2, 3, or more than 3 amino acids, and in which at least one of α and β contains a non-natural, or modified amino acid. For example, in a particular configuration, a plurality of binding targets may comprise at least 50 sequence contexts of epitope Θ, in which α comprises any post-translational modification of amino acid cysteine, and in which β can comprise any naturally-occurring, non-natural, or modified amino acid.

In some configurations, a plurality of binding targets may be provided with a plurality of unique binding targets, as determined by primary amino acid sequence, in which each binding target of the plurality of binding targets comprises a same terminal modified amino acid (e.g., modified for cleavage by an Edman-type degradation reaction). For example, a plurality of binding targets may be provided, in which each binding target comprises a cyclical phenylthiocarbamoyl Edman complex derivative at its N-terminus. In some configurations, a plurality of binding targets may be provided with a plurality of unique binding targets, in which each binding target of the plurality of binding targets comprises a same terminal modified amino acid (e.g., modified for cleavage by an Edman-type degradation reaction) in a different sequence context. For example, a plurality of binding targets may comprise at least about 10, 50, 100, 200, 400, 1000, 2000, 5000, 10000, 15000, 20000, 50000, 100000, 1000000, or more than 1000000 sequence contexts of structure X₁X₂*, where X₁ is any naturally-occurring, non-natural, or modified amino acid, and where X₂* is a cyclical phenylthiocarbamoyl Edman complex derivative of any naturally-occurring, non-natural, or modified amino acid.

In some cases, a binding target of a plurality of binding targets may comprise an amino acid sequence of a known protein (e.g., a partial amino acid sequence). In particular cases, a binding target of a plurality of binding targets may comprise a complete amino acid sequence of a known protein. In some configurations, a binding target of a plurality of binding targets may comprise a repeat of an epitope Θ (e.g., a peptide comprising a sequence ΘΘΘ, a peptide comprising a sequence ΘXΘXΘ, where X is a spacing moiety that can comprise amino acids and/or a polymer linker). In some configurations, a plurality of binding targets can comprise a plurality of binding targets, in which each binding target of the plurality of binding targets comprises an identical amino acid sequence. Alternatively, a plurality of binding targets can comprise a plurality of binding targets, in which the plurality of binding targets comprises two or more binding targets that differ with respect to amino acid sequence.

A plurality of binding targets may comprise a plurality of peptides, in which a peptide of the plurality of peptides is at least about 5, 10, 15, 20, 25, 30, 40, 50, 100, or more than 100 amino acids in length. Alternatively or additionally, an array of binding targets may comprise a plurality of peptides, in which a peptide of the plurality of peptides is no more than about 100, 50, 40, 30, 25, 20, 15, 10, 5, or less than 5 amino acids in length. A plurality of binding targets may comprise a plurality of peptides, in which each peptide is at least about 5, 10, 15, 20, 25, 30, 40, 50, 100, or more than 100 amino acids in length. Alternatively or additionally, a plurality of binding targets may comprise a plurality of peptides, in which each peptide is no more than about 100, 50, 40, 30, 25, 20, 15, 10, 5, or less than 5 amino acids in length.

In some configurations, each individual binding target of an array of binding targets may be separated on a solid support by an optically resolvable distance from any other binding target of the array of binding targets. In some cases, an array of binding targets may comprise a single-molecule array. Accordingly, a method of identifying a binding reagent from a library of binding reagents can comprise detecting binding of the binding reagent to a binding target of an array of binding targets at single-molecule resolution (e.g., detecting a signal from the binding reagent at a single address containing the binding target). An array of binding targets may comprise a plurality of addresses, each address containing one and only one immobilized binding target, in which the addresses have an average pitch as measured by the average separation between respective centerpoints of adjacent addresses.

Alternatively, an array of binding targets may be provided, in which binding targets of a plurality of binding targets are separated by an optically non-resolvable distance. An array of binding targets may comprise a plurality of addresses, in which each address comprises a plurality of binding targets (e.g., a protein microarray). An array address may comprise a plurality of binding targets, in which each binding target of the array address comprises the same primary amino acid structure. An array address may comprise a plurality of binding targets, in which each binding target of the array address comprises the same proteoform. An array address may comprise a plurality of binding targets, in which the array address comprises two or more binding targets having differing primary amino acid structures. An array address may comprise a plurality of binding targets, in which the array address comprises two or more binding targets having differing proteoforms.

A retaining component, such as a SNAP, may have any of a variety of sizes and shapes to accommodate use in a desired application. For example, a retaining component can have a regular or symmetric shape or, alternatively, it can have an irregular or asymmetric shape. The shape can be rigid or pliable. The size or shape of a SNAP or other retaining component can be characterized with respect to area (e.g. footprint), or volume. The size or shape of a SNAP or other retaining component can be smaller than an address in an array to which it will associate or attach. Optionally, the relative sizes and shapes of an individual retaining component and an address to which it will attach are configured to preclude more than one of the retaining components from occupying the address.

Optionally, a retaining component (e.g. SNAP) or population thereof has a minimum, maximum or average volume of at least about 1 micron³, 10 micron³, 100 micron³, 1 mm3 or more. Alternatively or additionally, a retaining component (e.g. SNAP) or population thereof has a minimum, maximum or average volume of no more than about 1 mm³, 100 micron³, 10 micron³, 1 micron³ or less. Optionally, the minimum, maximum or average area (e.g. footprint) for a retaining component (e.g. SNAP) is at least about 10 nm², 100 nm², 1 micron², 10 micron², 100 micron², 1 mm² or more. Alternatively or additionally, the minimum, maximum or average area for a retaining component (e.g. SNAP) footprint is at most about 1 mm², 100 micron², 10 micron², 1 micron², 100 nm², 10 nm², or less. The footprint of a retaining component (e.g. SNAP) may have a regular shape or an approximately regular shape, such as triangular, square, rectangular, circular, ovoid, or polygonal shape. An address of an array can have one of these shapes as well, whether or not the address has the same shape as a SNAP to which it attaches.

A structured nucleic acid particle (e.g. having origami or nanoball structures) may include regions of single-stranded nucleic acid, regions of double-stranded nucleic acid, or combinations thereof. For example, a SNAP can have a nucleic acid origami structure which includes a scaffold strand and a plurality of staple strands. The scaffold strand can be configured as a single, continuous strand of nucleic acid, and the staples can be formed by nucleic acid strands that hybridize, in whole or in part, with the scaffold strand.

In some configurations, a nucleic acid origami includes a scaffold composed of a nucleic acid strand to which a plurality of oligonucleotides is hybridized. A nucleic acid origami may have a single scaffold molecule or multiple scaffold molecules. A scaffold strand can be linear (i.e. having a 3′ end and 5′ end) or circular (i.e. closed such that the scaffold lacks a 3′ end and 5′ end). A scaffold strand can be derived from a natural source, such as a viral genome or a bacterial plasmid. For example, a nucleic acid scaffold can include a single strand of an M13 viral genome. In other configurations, a scaffold strand may be synthetic, for example, having a non-naturally occurring nucleotide sequence in full or in part. A scaffold nucleic acid can be single stranded but for a plurality of oligonucleotides hybridized thereto or short regions of internal complementarity. The size of a scaffold strand may vary to accommodate different uses. For example, a scaffold strand may include at least about 100, 500, 1000, 2500, 5000 or more nucleotides. Alternatively or additionally, a scaffold strand may include at most about 5000, 2500, 1000, 500, 100 or fewer nucleotides.

A nucleic acid origami can include one or more oligonucleotides that are hybridized to a scaffold strand. An oligonucleotide can include two sequence regions that are hybridized to a scaffold strand, for example, to function as a ‘staple’ that restrains the structure of the scaffold. For example, a single oligonucleotide can hybridize to two regions of a scaffold strand that are separated from each other in the primary sequence of the scaffold strand. As such, the oligonucleotide can function to retain those two regions of the scaffold strand in proximity to each other or to otherwise constrain the scaffold strand to a desired conformation. Two sequence regions of an oligonucleotide staple that bind to a scaffold strand can be adjacent to each other in the nucleotide sequence of the oligonucleotide or separated by a spacer region that does not hybridize to the scaffold strand.

An oligonucleotide can include a first sequence region that is hybridized to a complementary sequence of a nucleic acid origami and a second region that provides a “handle” or “linker” for attaching another moiety. Optionally, the moiety can be attached to an oligonucleotide that is complementary to the second region of the handle and the moiety can be attached to the nucleic acid origami via hybridization of the handle to the complementary oligonucleotide. The moiety can be a protein or other analyte, for example, when the SNAP is used to mediate attachment to an array. Other moieties that can be attached to a SNAP include, but are not limited to, a paratope, affinity reagent (e.g. antibody), or reactive moiety such as a click reagent.

Oligonucleotides can be configured to hybridize with a nucleic acid scaffold, another oligonucleotide, a staple oligonucleotide, or a combination thereof. One or more regions of an oligonucleotide that hybridizes to another sequence of a nucleic acid origami or other structured nucleic acid particle can be located at or near the 5′ end of the oligonucleotide, at or near the 3′ end of the oligonucleotide, or in a region of the oligonucleotide that is between the end regions. The oligonucleotides can be linear (i.e. having a 3′ end and a 5′ end) or closed (i.e. circular, lacking both 3′ and 5′ ends). An oligonucleotide that is included in a nucleic acid origami or other structured nucleic acid particle can have any of a variety of lengths including, for example, at least about 10, 25, 50, 100, 250, 500, or more nucleotides. Alternatively or additionally, an oligonucleotide may have a length of no more than about 500, 250, 100, 50, 25, 10, or fewer nucleotides. An oligonucleotide may form a hybrid of at least about 5, 6, 7, 8, 9, 10, 15, 20, 25, 50 or more consecutive or total base pairs with another nucleotide sequence of a nucleic acid origami. Alternatively or additionally, an oligonucleotide may form a hybrid of no more than about 50, 25, 20, 15, 10, 9, 8, 7, 6, 5, or fewer consecutive or total base pairs with another nucleotide sequence.

A retaining component may be provided with moieties that facilitate coupling with a surface of a solid support. The moieties can be configured to form a covalent interaction or a non-covalent interaction with the solid support. In an example, a retaining component may be provided with one or more nucleic acid strands that hybridize to an immobilized nucleic acid strand on a surface of a solid support by Watson-Crick hybridization. Preferably, a retaining component may be provided with a plurality of moieties that can bind to a surface of a solid support.

A structured nucleic acid particle (e.g., nucleic acid origami, or nucleic acid nanoball) may be formed by an appropriate technique including, for example, those known in the art. Nucleic acid origami can be designed, for example, as described in Rothemund, Nature 440:297-302 (2006), or U.S. Pat. Nos. 8,501,923 or 9,340,416, each of which is incorporated herein by reference. Nucleic acid origami may be designed using a software package, such as CADNANO (cadnano.org), ATHENA (github.com/lcbb/athena), or DAEDALUS (daedalus-dna-origami.org).

Other useful retaining components include artificial polymers. Artificial polymers can include polymers that are made by human activity rather than occurring naturally. For example, a polymer that is made at least in part by human activity or that includes at least one artificial moiety is referred to as an “artificial polymer.” In some cases the artificial polymers are configured as dendrons. Dendrons include at least one branched chain polymer. A branched chain polymer can include at least 1, 2, 3, 4, 5, 6, 8 or 10 branch points. Alternatively or additionally, a branched chain can include at most 10, 8, 6, 5, 4, 3, 2 or 1 branch points. A branch point is a covalent intersection between at least two chains. For example, at least 2, 3, 4, 5 or more chains can intersect at a branch point of a branched chain. Alternatively or additionally, at most 5, 4, 3 or 2 chains can intersect at a branch point of a branched chain. A polymer, whether branched or not, can include a single type of monomer subunit or multiple different types of monomer subunits. Accordingly, a polymer can include at least 1, 2, 3, 4, 5 or more different types of monomer subunits. Alternatively or additionally, a polymer can include at most 5, 4, 3, 2 or 1 different types of monomer subunits. A polymer having only one type of subunit in the network of covalent bonds is referred to as a “homopolymer.” In contrast, a “copolymer” includes two or more different types of subunits in the network of covalent bonds.

A retaining component that includes an artificial polymer can have a volume or footprint in a range set forth above for SNAPs. A retaining component can be further characterized in terms of molecular weight (or molecular weight distribution) in a desired size range. For example, the molecular weight, average molecular weight distribution, minimum molecular weight distribution or maximum molecular weight distribution can be at least 1 kDa, 2 kDa, 5 kDa, 10 kDa, 25 kDa, 50 kDa or more. Alternatively or additionally, the molecular weight, average molecular weight distribution, minimum molecular weight distribution or maximum molecular weight distribution can be at most 50 kDa, 25 kDa, 10 kDa, 5 kDa, 2 kDa, 1 kDa or less. A retaining component can be characterized in terms of radius of gyration. For example, the radius of gyration can be at least about 2 nm, 5 nm, 10 nm, 15 nm, 25 nm, 50 nm or more. Alternatively or additionally, retaining component can be configured to have a radius of gyration that is at most about 50 nm, 25 nm, 15 nm, 10 nm, 5 nm, 2 nm or less. An artificial polymer can be characterized in term of degree of polymerization (i.e. number of monomer subunits) present. For example, an artificial polymer can include at least 2, 10, 20, 30, 40, 50, 100, 200, 300 or more monomers. Alternatively or additionally, an artificial polymer can include at most 300, 200, 100, 50, 40, 30, 20, 10, or 2 monomers.

An artificial polymer can lack natural polymers or monomers found in natural polymers. For example, the skeletal structure of the artificial polymer can lack natural polymers or monomers. This can be the case whether or not the artificial polymer has attached moieties that include natural polymers or monomers. Examples of natural moieties that can be absent from an artificial polymer, for example in the skeletal structure include, but are not limited to, nucleic acids (e.g. DNA or RNA), nucleotides (e.g. deoxyribonucleotides or ribonucleotides), nucleosides (e.g. deoxyribonucleosides or ribonucleosides), proteins, amino acids, or sugars (e.g. saccharide monomers, monosaccharides, oligosaccharides, polysaccharides or glycans). An artificial polymer can optionally lack any polymer or monomer that is synthesized in vivo or that is capable of being synthesized in vivo. Alternatively, an artificial polymer can include natural moieties that are combined to form a non-naturally occurring molecule. For example, an artificial polymer can be composed of nucleic acid monomers or nucleic acid strands that form a non-naturally occurring nucleic acid dendrimer structure.

Particularly useful artificial polymers include, for example, poly(amidoamine) (PAMAM) dendrimer, poly(amidoamine) dendron, hyperbranched polymers such as linear and branched polyethyleneimine (PEI) and polypropyleneimine (PPI), star polymers, grafted polymers, peptide-based linear or branched dendrimers such as branched poly-L-lysine (PLL) and silane-cored dendrimer. Other useful artificial polymers include dendrimer nucleic acids having branching structures. See, for example, Liu et al., J. Mater. Chem. B 9:4991-5007 (2021) and Meng et al., ACS Nano 8:6171-6181 (2014), each of which is incorporated herein by reference. Examples of useful polymers are set forth in Tomalia, et al. J Polym Sci Part A: Polym Chem 40:2719-2728 (2002); Higashihara, et al. Polym J 44, 14-29 (2012); Gupta, et al. J. Phys. Chem. B 124, 20, 4193-4202 (2020); Ren, et al. Chem. Rev. 116, 12, 6743-6836 (2016); Chis, et al. Molecules 25 (17): 3982 (2020); Zheng, et al. Chem. Soc. Rev. 44, 4091-4130 (2015), or U.S. patent application Ser. No. 18/438,973, each of which is incorporated herein by reference.

A method of the present disclosure can include a step of coupling one or more proteins to a solid support or a surface thereof, for example, prior to performing a kinetic or thermodynamic assay, affinity reagent binding reaction or detection step set forth herein. The coupling of one or more proteins to a solid support may include covalent and/or non-covalent coupling. Covalent coupling of a protein to a solid support can include direct covalent coupling of the protein to the solid support (e.g., formation of coordination bonds) or indirect covalent coupling between a reactive functional group of the protein and a reactive functional group that is coupled to the solid support (e.g., a CLICK-type reaction). Non-covalent coupling can include the formation of any non-covalent interaction between a protein and a solid support, including electrostatic or magnetic interactions, or non-covalent bonding interactions (e.g., ionic bonds, van der Waals interactions, hydrogen bonding, etc.).

An array may be provided with a dynamic range of proteins. Dynamic range can refer to the ratio of abundance between a more populous protein species and a less populous protein species. A dynamic range can be a comprehensive measure (ratio of most populous protein species to least populous protein species) or a limited measure (ratio of a first protein species to a second protein species). An array of proteins may be provided with a dynamic range of at least about 10, 10², 10³, 10⁴, 10⁵, 10⁶, 10⁷, 10⁸, 10⁹, 10¹⁰, 10¹¹, 10¹², or more. Alternatively or additionally, an array of analytes may be provided with a dynamic range of no more than about 10¹², 10¹¹, 10¹⁰, 10⁹, 10⁸, 10⁷, 10⁶, 10⁵, 10⁴, 10³, 10², 10, or less.

An array can be formed by a process that includes a step of coupling proteins to addresses of the array. A protein may be coupled to an address reacting a coupling moiety of the analyte with a compatible coupling moiety of the address. In some cases where a protein is attached to a retaining component, a step of coupling the protein to the address may include coupling the retaining component to the address. Alternatively, a retaining component can be coupled to an address and then a protein can be coupled to the retaining component.

A method set forth herein can include performing a kinetic assay. The kinetic assay can be configured to determine the rate at which an affinity reagent binds to a protein to form a complex, the rate at which an affinity reagent dissociates from the complex, or both. The data can be used to derive kinetic or equilibrium constants such as to association rate constant (kₒₙ), dissociation rate constant (k_{off}), equilibrium dissociation constant (K_{D}) or equilibrium association constant (K_{A}). A kinetic assay that is configured to determine association rate can be initiated by contacting affinity reagents with proteins under conditions in which the affinity reagents and proteins are permitted to bind to each other to form complexes. Association rates can be determined by performing the assay in a first mode whereby a protein sample, such as a single molecule-resolved protein array, is contacted with a series of fluids containing affinity reagents, wherein each of the fluids has a different concentration of the affinity reagents and the duration of contact is constant for all of the fluids. In a second mode of the association rate assay, a protein sample, such as a single molecule-resolved protein array, can be contacted with a series of fluids containing affinity reagents, wherein each of the fluids has identical concentration of the affinity reagents and the affinity reagents are contacted with the proteins for different durations. In either mode, interactions between the affinity reagents and proteins can be observed after each fluidic delivery, for example, by detecting gain of signal at protein addresses due to association with labeled affinity reagents. In this way, an increase in the number of complexes can be measured over time, during pre-equilibrium conditions, to determine an association rate. Equilibrium is achieved when the rate of at which complexes are formed between proteins and affinity reagents is equal to the rate of dissociation for the complexes. Pre-equilibrium occurs when the ratio of formed complexes to non-complexed components is lower than the ratio at equilibrium.

Dissociation rates can be determined by measuring a decrease in protein-affinity reagent complexes over time, for example, during a post-equilibrium condition. Complexes can be formed by contacting a protein sample, such as a single molecule-resolved protein array, with fluid phase affinity reagents and allowing binding to occur until equilibrium has been reached. The fluid and its contents of unbound affinity reagents can then be removed from contact with the proteins to create a disequilibrium, whereby the affinity reagents dissociate from the proteins. The reaction can be monitored for dissociation of the complexes, for example, as loss of signal at protein addresses in the array due to dissociation of labeled affinity reagents.

In some cases, an assay can be configured for equilibrium analysis. For example, various concentrations of labeled affinity reagents can be incubated with a protein sample, such as a single molecule-resolved protein array and, after the reaction has been allowed to achieve equilibrium, the quantity of signal measured from each protein (e.g. signal measured from each address in a protein array) can be detected to determine the extent of binding to the labeled affinity reagents. Achievement of equilibrium can be determined in a number of ways. For example, equilibrium can be observed as saturation in the trend of increasing signal intensity to increasing affinity reagent concentration. Whether equilibrium has been achieved at a particular affinity reagent concentration and reaction duration can be determined by (i) creating an equilibrium binding reaction under the conditions and using a labeled affinity reagent, (ii) replacing unbound labeled affinity reagents in the binding mixture with unlabeled affinity reagents, and (iii) detecting the mixture to determine if there is any change in the amount of labeled affinity reagents in the complex. For example, the experiment can be performed using a single molecule-resolved protein array and the addresses of the array can be monitored for a change in signal upon addition of unlabeled affinity reagents. Different ratios of labeled and unlabeled affinity reagents can be evaluated in this way to determine an equilibrium binding constant for the reaction. Results can be evaluated using a Langmuir binding analysis.

Measurement of the binding kinetics of an affinity reagent may be carried out according to the intended assay for the affinity reagent. For example, some assays may be performed under non-equilibrium conditions that utilize rinse steps to remove unbound affinity reagents after incubation with binding targets. Accordingly, methods of the present disclosure may introduce rinse steps or other conditions that produce a state of non-equilibrium, thereby facilitating measurement of association and/or dissociation rates under the non-equilibrium condition. In another example, some assays may be performed under equilibrium conditions, in which affinity reagent binding to binding targets is detected in the presence of unbound affinity reagents. Accordingly, methods of the present disclosure may omit rinse steps or other conditions that produce a state of non-equilibrium, thereby facilitating measurement of association and/or dissociation rates under the equilibrium condition.

Methods of the present disclosure may be amenable to techniques that facilitate single-analyte detection of affinity reagent binding under equilibrium conditions. In some cases, arrays of binding targets may be provided, in which each individual binding target is isolated in a well or depression (e.g., a nanowell, a microwell). Preferably, an array may comprise a plurality of wells or depressions, in which each well or depression comprises only one binding target. Alternatively, an array may comprise a plurality of wells or depressions, in which each well or depression comprises a plurality of binding targets. If binding targets are immobilized at the bottom surface of a well or depression, then illumination of the bottom surface of the well or depression may transmit light only into the lower portion of the well or depression, thereby facilitating detection of labeled affinity reagent that are bound to an immobilized binding target without producing significant background from unbound affinity reagents in the well or depression. An advantageous configuration of an array may comprise an optically-transmitting substrate (e.g., glass, quartz, fused silica, etc.) covered by a metal layer, in which the metal layer is patterned into a plurality of wells or depressions. Preferably, the wells or depressions may have an average diameter or width that is less than the wavelength of light utilized to illuminate fluorescent or luminescent labels in the well (e.g., less than about 500 nanometers (nm), 400 nm, 300 nm, 250 nm, 200 nm, 150 nm, 100 nm, or 50 nm). Binding targets may be immobilized to a surface of the optically-transmitting material that is exposed in the bottoms of the wells or depressions. Light may be passed through the optically-transmitting substrate, thereby illuminating the bottom portions of the wells or depressions comprising immobilized binding targets.

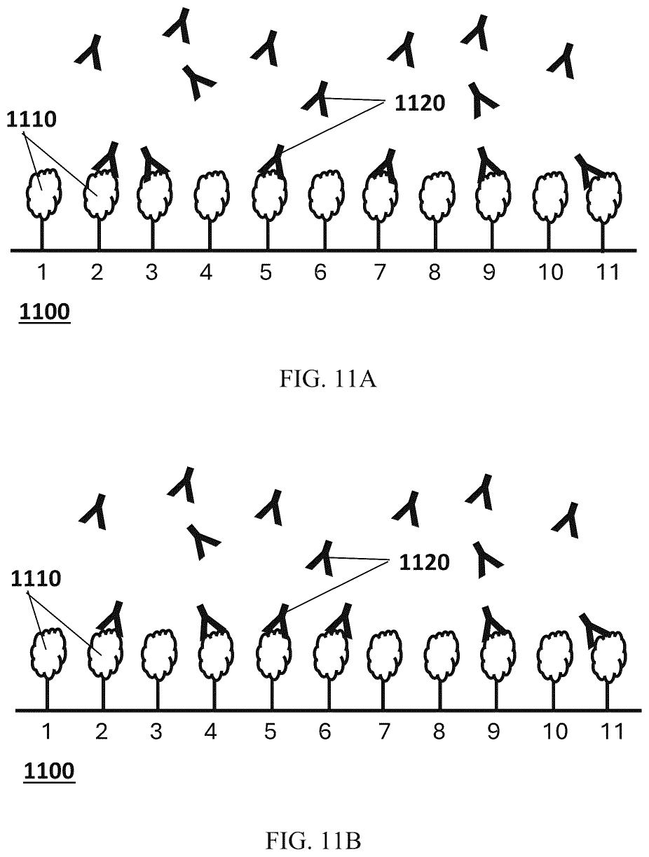

FIGS. 11A-11B illustrate portions of a system that may be useful for measuring binding kinetics and/or equilibrium at single-analyte resolution. FIG. 11A depicts a configuration of a system at a first timepoint, in which the system comprises a solid support 1100 containing a plurality of individually observable addresses (1 to 11), in which each address contains only one analyte 1110 of a plurality of analytes immobilized on the solid support 1100. The solid support is contacted with a pool of affinity reagents 1120. A first fraction of affinity reagents 1120 of the pool of affinity reagents 1120 are in a fluid phase (i.e., unbound to analytes 1110) and a second fraction of affinity reagents 1120 of the pool of affinity reagents 1120 are bound to analytes 1110. It can be observed (e.g., by fluorescent microscopy or other single-analyte observation methods) that analytes 1110 at addresses 2 3, 5, 7, 9, and 11 are bound to affinity reagents 1120. FIG. 11B depicts a configuration of the system at a second timepoint, in which the total quantity of analytes 1110 bound by affinity reagents 1120 is substantially stable between the first and second timepoints (i.e., the system is at binding equilibrium) but the specific analytes 1110 bound by affinity reagents 1120 has changed due to association and dissociation of affinity reagents 1120. It can be observed that analytes 1110 at addresses 2, 5, 9, and 11 remain bound to affinity reagents 1120, analytes 1110 at addresses 3 and 7 have dissociated from affinity reagents 1120, and analytes 1110 at addresses 4 and 6 have associated with affinity reagents 1120. Additional observations of the system can be made, and the observations can be utilized to calculate population-wide measures of the length of time that an affinity reagent 1120 remains associated to an analyte 1110 and/or the length of time that an analyte 1110 remains unbound by an affinity reagent 1120. The population-wide measures of association time and/or dissociation time can be utilized to calculate an association rate constant and/or a dissociation rate constant for the affinity reagents 1120 with the analytes 1110.

Observation of association and/or dissociation of affinity reagents from binding targets at single-analyte resolution may be performed at a sampling frequency that allows binding dynamics of a system to be observed. In particular, it may be advantageous to have a sampling frequency that exceeds a maximum expected association rate or dissociation rate of affinity reagents with binding targets. For example, if affinity reagents are expected to associate or dissociate from binding targets at the rate of 1/s, it may be preferable to have a measurement frequency of at least 2/s, 5/s, 10/s, 20/s, 50/s, 100/s, or more than 100/s (i.e., a sampling rate of at least about 2×, 5×, 10×, 20×, 50×, 100×, or more than 100× the association or dissociate rate). Accordingly, depending upon the association rates and/or dissociation rates of affinity reagents with binding targets, elapsed time between observation timepoints may be at about 0.001 seconds(s), 0.01 s, 0.1 s, 0.2 s, 0.5 s, 1 s, 2 s, 5 s, 10 s, 30 s, 1 minute (min), 5 mins, 10 mins, or more than 10 mins apart. Alternatively or additionally, elapsed time between observation timepoints may be at least about no more than about 10 mins, 5 mins, 1 min, 30 s, 10 s, 5 s, 2 s, 1 s, 0.5 s, 0.2 s, 0.1 s, 0.01 s, 0.001 s, or less than 0.001 s apart.

Measurement of association rate under equilibrium conditions may be particularly advantageous. Measurement of association rate under non-equilibrium conditions may require repeated cycles of incubating affinity reagents with binding targets, with each cycle requiring a different incubation time as well as a rinse step to remove unbound affinity reagents. In the equilibrium-based methods set forth herein, binding of affinity reagents to binding targets can be observed until a stable population of bound affinity reagents is achieved.

An assay that is used in accordance with the present disclosure can be configured for single molecule-resolved detection. This can be achieved for example by attaching one of the complex forming components to a solid support and contacting the solid support with a fluid containing the other component for forming the complex. For example, a protein can be immobilized on a solid support and then contacted with a fluid phase containing affinity reagents. Aspects of the assay are exemplified herein in the context of immobilized proteins and fluid phase affinity reagents. However, those skilled in the art will recognize that the teachings herein can be extended to a format in which affinity reagents are immobilized and contacted with fluid phase proteins. A plurality of proteins can be provided in an array format, and each of the proteins in the array can be individually resolved from every other protein in the array. As such, binding of each protein to an affinity reagent can be measured, thereby providing single molecule-resolved detection. However, population dynamics can be determined from a combination of single molecule-resolved measurements. For example, the same species of proteins can be attached to each of the respective addresses and the addresses can be contacted with a fluid containing a plurality of affinity reagents that form complexes with the proteins. By counting the number of addresses that form a complex over time, an association rate can be determined for the population of proteins on the array. Similarly, the number of addresses from which affinity reagents dissociate can be counted to determine a dissociation rate. An advantage of monitoring single molecule-resolved proteins is that subpopulations of proteins can be identified based on different apparent association rates or dissociation rates. The observations can be used to identify population dynamics that would otherwise be averaged out in assays that detect ensemble-based addresses in arrays or that detect bulk solutions.

Assays are exemplified herein in the context of using fluid phase affinity reagents having optical labels (e.g. luminescent labels) and detecting optical signals (e.g. luminescence) at array addresses where the labeled affinity reagents have bound to a resident protein. For configurations in which the affinity reagents reside at the addresses and the proteins are in solution phase, the labelling scheme can be reversed (i.e. the proteins can bear the labels). Moreover, it will be understood that any of a variety of labels and detectors of their signals can be used as will become evident to those skilled in the art based on the teachings herein.

An assay that is configured for determining association rates between proteins and affinity reagents can include the following steps: (i) contacting the array with a set of affinity reagents, wherein the affinity reagents have optical labels, (ii) detecting binding of the affinity reagents to proteins at addresses of the array, wherein the detecting includes acquiring optical signals from the optical labels at respective addresses of the array, wherein the respective addresses are individually resolved, and (iii) removing affinity reagents from the array, wherein steps (i) through (iii) are repeated for a plurality of cycles, each of the cycles using another set of affinity reagents instead of the set of affinity reagents, wherein affinity reagent species composition of the set of affinity reagents is identical to the other set of affinity reagents, and wherein concentration of the set of affinity reagents differs from concentration of the other set of affinity reagents.

A set of affinity reagents can be contacted with an array using a fluidic technique that is appropriate to the hardware used. For example, fluid phase affinity reagents can be delivered by dipping the array in the fluid, pipetting the fluid onto the array surface, or flowing the fluid across the array surface. In particular embodiments, the array is contained in a flow cell having an ingress through which fluid is delivered and an egress through which fluid is removed.

A set of affinity reagents that is delivered to an array can include a quantity (e.g. concentration) of affinity reagents that is known or suspected to facilitate binding to the proteins in the array. Typically, the set of affinity reagents will include a single species of affinity reagent. This is beneficial, for example, when using the assay to evaluate binding properties for a particular species of affinity reagent. However, in some cases a mixed pool of affinity reagent species can be present in the set. Optionally, the different species of affinity reagent can be distinguishably labeled. As such, addresses that bind to different species can be distinguished to allow characterization of binding properties for each respective species of affinity reagent in the set.

Binding of an affinity reagent to a protein at a given address can be detected as signal emanating from the address, wherein the signal is produced by a label that is attached to the affinity reagent when bound to the protein. Absence of the signal at other addresses indicates that labeled affinity reagent has not bound at those other addresses. Affinity reagents and labels are set forth in further detail below. Detection can be carried out as set forth in further detail below.

Following detection, affinity reagents can be removed from contact with proteins. For example, affinity reagents can be removed from contact with an array of proteins after detection of binding between the affinity reagents and proteins. An assay of the present disclosure can be carried out in multiple cycles, each cycle including two or more steps. An assay of the present disclosure can include at least 2, 3, 4, 5, 6, 7, 8, 9, 10 or more cycles. Alternatively or additionally, an assay can include at most 10, 9, 8, 7, 6, 5, 4, 3, 2 or 1 cycles. Any number or combination of steps set forth herein can be included in the cycles. For example, each cycle can include steps of contacting an array with a set of affinity reagents and detecting binding of the affinity reagents to addresses of the array. As such, an assay of the present disclosure can include a cycle in which a set of affinity reagents is contacted with an array and a subsequent cycle in which another set of affinity reagents is contacted with the array. Two or more sets of affinity reagents that are used in respective cycles of an assay can contain the same species of affinity reagent. This can be useful for evaluating binding characteristic for the species of binding agent by comparing binding results under different conditions. Alternatively, two or more sets of affinity reagents that are used in respective cycles of an assay can differ with regard to affinity reagent compositions. For example, affinity reagents can differ with regard to the number of paratopes or affinity moieties that are present in the affinity reagents used for respective cycles. Other compositional differences for affinity reagents can also occur between cycles including, but not limited to, different numbers of labels, different species of labels, different retaining components, different species of paratopes or affinity moieties, or others set forth herein.

In some configurations of a cyclic assay, the two or more sets of affinity reagents that are used in respective cycles can be contacted with the array under substantially identical conditions. This can be done, for example, to provide replicate measures. The replicate measures can be useful for performing statistical analysis of the data, for example to determine error rates, variation, dispersion, significance, or the like. In other configurations, the two sets of affinity reagents can be contacted with the array under differing conditions. Conditions that can differ include, for example, quantity (e.g. concentration) of the affinity reagents, duration of contact between an array and fluids containing the affinity reagents prior to detection, pH, ionic strength, temperature, signal gain for a detector, duration of signal acquisition by a detector, or number of signal acquisitions performed by a detector.

Optionally, an assay of the present disclosure can include a step of removing unbound affinity reagents from contact with an array prior to detecting affinity reagents that are bound to the array. For example, an assay can include steps of (i) contacting the array with a set of affinity reagents, wherein the affinity reagents have optical labels, (ii) washing the array to remove unbound affinity reagents of the set, (iii) detecting affinity reagents bound to proteins at addresses of the array, wherein the detecting includes acquiring optical signals from the optical labels at respective addresses of the array, wherein the respective addresses are individually resolved, and (iv) removing unbound affinity reagents from contact with the array, wherein steps (i) through (iv) are repeated for a plurality of cycles, each of the cycles using another set of affinity reagents instead of the set of affinity reagents, wherein affinity reagent species composition of the set of affinity reagents is identical to the other set of affinity reagents, and wherein concentration of the set of affinity reagents differs from concentration of the other set of affinity reagents. For configurations that use a wash step, wash conditions can differ between cycles. For example, conditions can differ with respect to chemical composition of the wash fluids, duration of the wash steps, temperature during the wash steps, pH of the wash fluids, ionic strength of the wash fluids, volume of wash fluids contacted with the array, or the like. Alternatively, identical wash conditions can be used across multiple cycles.

An assay that is configured for determining dissociation rates for complexes formed between proteins and affinity reagents can include the following steps: (b) performing an assay, including (i) contacting the array with a set of affinity reagents, wherein the affinity reagents include optical labels, and wherein the affinity reagents bind to proteins at addresses of the array, (ii) detecting proteins at addresses of the array that are bound to affinity reagents of the set, wherein the detecting includes acquiring optical signals from the optical labels at respective addresses of the array, wherein the respective addresses are individually resolved, and (iii) repeating step (ii) for a plurality of cycles, thereby detecting a decay in optical signals at the respective addresses of the array. Optionally, a wash step can be performed after step (i) and prior to step (ii) in order to remove excess or unbound affinity reagents. In a typical configuration, the wash step can be carried out in a first cycle and need not be repeated for subsequent cycles. However, multiple wash steps can be carried out if desired.

An assay for determining dissociation rates can use affinity reagents and delivery conditions set forth above in context of determining association rates. Dissociation of an affinity reagent from a protein at a given address can be detected as a loss of signal emanating from the address, wherein the signal is produced by a label that is attached to the affinity reagent when bound to the protein. Retention of signal at other addresses indicates that labeled affinity reagent has not dissociated at those other addresses.

Detection can be carried out in a continuous mode, for example, acquiring signals from addresses of an array at a particular frame rate (e.g. a movie). Alternatively, detection can be carried out by acquiring signals at discrete timepoints. The time points of frame rate can be on the order of nanoseconds (e.g. 1 to 999 nanoseconds per acquisition), microseconds (e.g. 1 to 999 microseconds per acquisition), milliseconds (e.g. 1 to 999 milliseconds per acquisition), seconds (e.g. 1 to 999 seconds per acquisition), or minutes (e.g. 1 to 59 seconds per acquisition). Each signal acquisition can be considered a cycle of a kinetic determination of dissociation rate. The number of cycles can include at least 2, 3, 4, 5, 6, 7, 8, 9, 10, 20, 50, 100, 500, 1000 or more cycles. Alternatively or additionally, the number of cycles can include at most 1000, 500, 100, 50, 20, 10, 9, 8, 7, 6, 5, 4, 3, 2 or 1 cycles. Detection cycles may be paced according to a constant or varying time interval. A next detection cycle may occur at least about 1 microsecond (μs), 1 millisecond (ms), 10 ms, 100 ms, 1 second(s), 5 s, 10 s, 15 s, 30 s, 1 minute (min), 5 mins, 10 mins, or more than 10 mins after a first detection cycle. Alternatively or additionally, a next detection cycle may occur no more than about 10 mins, 5 mins, 1 min, 30 s, 15 s, 10 s, 5 s, 1 s, 100 ms, 10 ms, 1 ms, 1 μs, or less than 1 μs after a first detection cycle. Detection may be characterized by an acquisition rate of detection events. For example, a detection device may detect signals from bound affinity reagents 10 times per second, thereby having an acquisition rate of 10 hertz (Hz). A detection device may have an acquisition rate of at least about 0.001 Hz, 0.01 Hz, 0.1 Hz, 0.5 Hz, 1 Hz, 2 Hz, 5 Hz, 10 Hz, 50 Hz, 100 Hz, 500 Hz, 1000 Hz, or more than 1000 Hz. Alternatively or additionally, a detection device may have an acquisition rate of no more than about 1000 Hz, 500 Hz, 100 Hz, 50 Hz, 10 Hz, 5 Hz, 2 Hz, 1 Hz, 0.5 Hz, 0.1 Hz, 0.01 Hz, 0.001 Hz, or less than 0.001 Hz.

An assay that is configured for determining both association rates and dissociation rates for proteins and affinity reagents can include the following steps (b) performing an assay, including (i) contacting the array with a set of affinity reagents, wherein the affinity reagents have optical labels, (ii) detecting binding of the affinity reagents to proteins at addresses of the array, wherein the detecting includes acquiring optical signals from the optical labels at respective addresses of the array, wherein the respective addresses are individually resolved, (iii) removing affinity reagents from the array, wherein steps (i) through (iii) are repeated for a plurality of cycles, each of the cycles using another set of affinity reagents instead of the set of affinity reagents, wherein affinity reagent species composition of the set of affinity reagents is identical to the other set of affinity reagents, and wherein concentration of the set of affinity reagents differs from concentration of the other set of affinity reagents, (iv) contacting the array with a further set of affinity reagents, wherein affinity reagent species composition of the set of affinity reagents is identical to the further set of affinity reagents, and wherein affinity reagents of the further set include optical labels, (v) detecting proteins at addresses of the array that are bound to affinity reagents of the further set, wherein the detecting includes acquiring optical signals from the optical labels at respective addresses of the array, wherein the respective addresses are individually resolved, and (vi) repeating step (v) for a plurality of cycles, thereby detecting a decay in optical signals at the respective addresses of the array.

Conditions and reagents for an assay that is configured for determining both association rates and dissociation rates can be selected from those set forth above in the context of either an assay for determining association rates or an assay for determining dissociation rates. For example, steps (i) through (iii) in the preceding assay can be repeated for any number of cycles, and step (v) in the preceding assay can be repeated for any number of cycles. The number of cycles used for steps (i) though (iii) can be the same as the number of cycles for step (v), but the number of cycles need not be the same. Typically, the sets of affinity reagents used for all steps will include identical species of affinity reagents. However, different species can be used if desired. Conditions can be changed between cycles and can include, for example, the conditions exemplified above in the context of assays for determining association rates or assays for determining dissociation rates.

A method of the present disclosure can include a step of determining an association rate between affinity reagents and proteins based on an assay, determining a dissociation rate between affinity reagents and proteins based on an assay, or determining both. Association rates and dissociation rates can be determined from a trend in the change of signal detected from a particular address of an array over a plurality of cycles in an assay. The use of an array of proteins allows such a trend to be determined for a plurality of addresses in the array. Data from the plurality of addresses can be combined in various ways. In a first configuration, a trend in the change of signal can be independently determined for each address in the plurality of addresses. The trends can then be combined, for example, to obtain a more accurate kinetic rate or a more statistically robust kinetic rate than would be determined from only one of the addresses. In a second configuration, signals from a plurality of addresses (or derivative data thereof) can be combined for each cycle, and then a trend in the change of the combined signals can be determined. Again, the trend can be used to obtain a more accurate kinetic rate or a more statistically robust kinetic rate than would be determined from only one of the addresses.

Any of a variety of kinetic measures can be determined from an assay set forth herein. Exemplary measures include, but are not limited to, an association rate constant (kₒₙ), dissociation rate constant (k_{off}), equilibrium association constant (K_{A}), or equilibrium dissociation constant (K_{D}). See, for example, Segel, Enzyme Kinetics John Wiley and Sons, New York (1975), which is incorporated herein by reference in its entirety. In some cases, a proxy association rate or proxy dissociation rate is determined. The rates are referred to as proxy rates since the measures may be influenced by factors other than association and dissociation. For example, an apparent association rate for binding partners may be slower than the actual association rate due to unobserved binding occurring during a delay between contacting the binding partners and detection. Similarly, an apparent dissociation rate for binding partners may be slower than the actual dissociation rate due to unobserved dissociation occurring during a delay between removing unbound species and detection. Delays can occur due to duration for mixing, duration of a wash step or both.

Signal data (or derivative data thereof) can be combined to provide an average kinetic rate, median kinetic rate or mean kinetic rate. Statistical measures that can be determined from the data include, for example, determining statistical variation, standard deviation, coefficient of variation, dispersion, probability distribution, frequency distribution or any statistical analysis known by a skilled artisan to be applicable. The number of addresses that are combined to determine a kinetic rate or to perform a statistical analysis can include at least 1, 2, 3, 4, 5, 10, 25, 50, 100, 250, 500, 1×10³, or more addresses. The addresses can include a subset of the addresses in an array or all addresses in an array.

In some cases, a plurality of addresses that is evaluated in an assay can include two or more subsets of addresses, wherein the subsets differ with respect to observed kinetic rates. For example, a first subset of addresses can be observed to have an association rate that is greater than the association rate observed for a second subset of addresses. In another example, a first subset of addresses can be observed to have a dissociation rate that is greater than the dissociation rate observed for a second subset of addresses. Signals can be combined for respective subsets of addresses, for example, as set forth herein above. Data obtained from respective subsets of addresses can be analyzed to determine kinetic rates or statistical measures, for example, as set forth herein above. An observation of different association rates and/or dissociation rates for respective subsets of addresses in an array can be used to identify affinity reagents that differentially bind to subsets of proteins. The subsets of different proteins can be identified, for example, to determine a characteristic of the proteins that impacts binding. The characteristics can include, for example, different degrees of denaturation, differential proteolysis, presence or absence of post-translationally modified amino acid residues, number of post-translationally modified amino acid residues, different locations for chemical moieties that attach the proteins to the addresses or the like. In some cases, it may be desirable to identify or select an affinity reagent that is less prone, or not prone, to having different association rates and/or dissociation rates for respective subsets of addresses in an array.

Methods of the present disclosure may be readily multiplexed. A method may be multiplexed with respect to binding targets on an array of binding targets, thereby facilitating simultaneous measurement of association and/or dissociation rates to each of the unique binding targets of the array of binding targets. For example, an array may comprise two or more structurally unique proteins, in which an association rate or a dissociation rate of an affinity reagent is measured with respect to each of the two or more structurally unique proteins. A method may be multiplexed with respect to affinity reagents, thereby facilitating simultaneous measurement of association and/or dissociation rates of each distinguishable affinity reagent to one or more binding targets. For example, two pools of affinity reagents may be contacted to an array of binding targets, in which the affinity reagents of the two pools of affinity reagents are distinguishable by differing detectable labels. Accordingly, respective binding of affinity reagents from each pool to binding targets can be detected by distinguishable signals from the affinity reagents of the two pools of affinity reagents.

Any of a variety of affinity reagents can be used in a composition or method set forth herein. An antibody is a particularly useful affinity reagent and can include any antigen-binding molecule or molecular complex having at least one complementarity determining region (CDR) that binds to or interacts with a particular antigen with high affinity. An antibody can include four polypeptide chains: two heavy chains (HC1 and HC2) and two light chains (LC1 and LC2). HC1 and HC2 can be covalently connected by one, two or more disulfide bonds. HC1 can be covalently connected to LC1 by at least one disulfide bond. HC2 can be covalently connected to LC2 by at least one disulfide bond. Each heavy chain can include a heavy chain variable region (V_{H}) and a heavy chain constant region (C_{H}). The heavy chain constant region can include three domains, C_{H1}, C_{H2} and C_{H3}. Each light chain can include a light chain variable region (V_{L}) and a light chain constant region (C_{L}). The V_{H} and V_{L} regions can further include regions of hypervariability, termed complementarity determining regions (CDRs), interspersed with regions that are more conserved, termed framework regions (FR). Each V_{H} and V_{L} can include three CDRs and four FRs, arranged from amino-terminus to carboxy-terminus in the following order: FR1, CDR1, FR2, CDR2, FR3, CDR3, FR4.

An antibody can include all elements of a full-length antibody, such as those enumerated above. However, an antibody need not be full length and functional fragments can be particularly useful. The term “antibody” as used herein encompasses full length antibodies and functional fragments thereof. A functional fragment can be naturally occurring, enzymatically obtainable, synthetic, or genetically engineered. An antibody can be obtained using any suitable technique such as proteolytic digestion or recombinant genetic engineering techniques involving the manipulation and expression of DNA encoding one or more antibody domains. Such DNA is readily available, for example, from commercial sources, DNA libraries (e.g., phage-antibody libraries), or can be synthesized. The DNA may be manipulated chemically or by using molecular biology techniques, for example, to arrange one or more variable and/or constant domains into a suitable configuration, or to introduce codons, introduce cysteine residues, remove cysteine residues, modify, add or delete other amino acids, etc.

A functional fragment of an antibody can include any fragment that is capable of binding to an epitope with a detectable affinity, such as a Fab, Fab′, F(ab′)₂, Fd, Fv, dAb, single-chain variable (scFv), di-scFv, tri-scFv, microantibody, or minimal recognition unit consisting of the amino acid residues that mimic the hypervariable region of an antibody (e.g., an isolated complementarity determining region (CDR) such as a CDR3 peptide). Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains can also be useful.

A functional fragment of an antibody will typically include at least one variable domain. The variable domain may be of any size or amino acid composition and will generally include at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} Or V_{L}-V_{L} dimers. Alternatively, a functional fragment of an antibody may contain a monomeric V_{H} or V_{L} domain.

In particular configurations, a functional fragment of an antibody contains at least one variable domain covalently connected to at least one constant domain. Non-limiting, exemplary configurations of variable and constant domains that may be found within an antigen-binding fragment of an antibody of the present disclosure include: (i) V_{H}-C_{H1}; (ii) V_{H}-C_{H2}; (iii) V_{H}-C_{H3}; (iv) V_{H}-C_{H1}-C_{H2}; (V) V_{H}-C_{H1}-C_{H2}-C_{H3}; (vi) V_{H}-C_{H2}-C_{H3}; (vii) V_{H}-C_{L}; (viii) V_{L}-C_{H1}; (ix) V_{L}-C_{H2}; (x) V_{L}-C_{H3}; (xi) V_{L}-C_{H2}; (xii) V_{L}-C_{H1}-C_{H2}-C_{H3}; (xiii) V_{L}-C_{H2}-C_{H3}; and (xiv) V_{L}-C_{L}. In any configuration of variable and constant domains, including any of the exemplary configurations listed above, the variable and constant domains may be either directly connected to one another or may be connected by a full or partial hinge or linker region. A hinge region may consist of at least 2 (e.g., at least 5, 10, 15, 20, 40, 60 or more) amino acids which result in a flexible or semi-flexible linkage between adjacent variable and/or constant domains in a single polypeptide molecule. Moreover, an antigen-binding fragment of an antibody may include a homo-dimer or hetero-dimer (or other multimer) of any of the variable and constant domain configurations listed above in non-covalent association with one another and/or with one or more monomeric V_{H} or V_{L} domain (e.g., by disulfide bond(s))

Other useful affinity reagents include, but are not limited to, affibodies, affilins, affimers, affitins, alphabodies, anticalins, avimers, DARPins, monobodies, nanoCLAMPs, nucleic acid aptamers, peptide aptamers, lectins or functional fragments thereof.

In some configurations of the methods, compositions or systems set forth herein, two or more affinity reagents can be present as moieties of a multimeric affinity reagent. For example, an affinity reagent can include two or more affinity moieties, wherein the affinity moieties are selected from an affinity reagent set forth herein or known in the art. Two or more affinity moieties can be combined via attachment to any of a variety of retaining components including, for example, a structured nucleic acid particle (SNAP), nucleic acid origami, artificial polymer or particle. Other particles (e.g. particles composed of solid support material set forth herein or known in the art) or substances, such as those set forth herein in the context of mediating attachment of a protein to a solid support, can be used as retaining components for affinity reagents. The presence of multiple affinity moieties in an affinity reagent can provide increased binding strength, for example, due to increased avidity as compared to any one of the affinity moieties when used as an individual affinity reagent. In some configurations an affinity reagent can include at least 2, 3, 4, 5, 6, 7, 8, 9, 10, 15, 20 or more affinity moieties. Alternatively or additionally, an affinity reagent can include at most 20, 15, 10, 9, 8, 7, 6, 5, 4, 3, 2 or fewer affinity moieties. It may be convenient to characterize an affinity reagent with respect to the number of paratopes it includes. For example, an affinity reagent can include at least 2, 3, 4, 5, 6, 7, 8, 9, 10, 15, 20 or more paratopes. Alternatively or additionally, an affinity reagent can include at most 20, 15, 10, 9, 8, 7, 6, 5, 4, 3, 2 or fewer paratopes. Typically, the affinity moieties or paratopes that are present in an affinity reagent will be structurally identical. For example, a plurality of antibodies in an affinity reagent can have identical amino acid sequences. Whether or not a plurality of affinity moieties or a plurality of paratopes include structurally identical members, the members can recognize the same epitopes. In some cases, the members can recognize the same epitopes with substantially the same binding strength. It will be understood, however, that in some cases an affinity reagent can include two or more affinity moieties having different structures and different binding affinities compared to each other. Similarly, an affinity reagent can include two or more paratopes having different structures and different binding affinities compared to each other.

In some configurations of the methods, compositions or systems set forth herein, an affinity reagent can include a plurality of labels. The labels can produce substantially identical signals, for example, due to the labels having identical structures. In some cases, the labels need not be structurally identical but may nevertheless produce signals that are indistinguishable using a given detector. For example, two luminophores may have different structure but may produce overlapping emission signals at a wavelength that is used for detection in a method set forth herein. The presence of multiple labels can be beneficial for increasing signal to noise compared to affinity reagents having only a single label. This can be especially helpful for use in an assay that is configured for single-protein resolution.

Optionally, an affinity reagent can include at least 1, 2, 3, 4, 5, 6, 7, 8, 9, 10, 15, 20, 25 or more labels. Alternatively or additionally, an affinity reagent can include at most 25, 20, 15, 10, 9, 8, 7, 6, 5, 4, 3, 2 or 1 label. Again, the labels can be the same or different with regard to structure or function. One or more labels can be attached to an affinity reagent via a retaining component, such as a retaining component that is also attached to a plurality of affinity moieties. One or more labels can be attached to other moieties of an affinity reagent. For example, label(s) can be attached to one or more affinity moieties of an affinity reagent. The presence of multiple labels on an affinity reagent can provide increased signal to noise and can thus increase sensitivity of detection to accommodate single-molecule resolved detection in a method set forth herein.

Exemplary labels and detectable signals they produce include, without limitation, optical labels such as luminophores (e.g. fluorophores) which emit photons at particular wavelengths when excited by radiation and which can be distinguished due to luminescence lifetime or polarization. Other useful optical labels include chromophores which absorb radiation at particular wavelengths and nanoparticles which can interact with light to produce signals such as photon emissions or light scatter. Other labels include heavy atoms, radioactive isotopes, mass labels, charge labels, spin labels, nucleic acids having particular sequence, receptors, ligands, or the like. Signals that can be detected from labels in methods set forth herein include, for example, optical signals such as absorbance of radiation, luminescence emission, luminescence lifetime, luminescence polarization, fluorescence fluorescence lifetime, fluorescence polarization, or the like; Rayleigh and/or Mie scattering; binding affinity for a ligand or receptor; magnetic properties; electrical properties; charge; mass; radioactivity, nucleotide sequences detected via nucleic acid sequencing platforms, sequence specific hybridization of nucleic acid labels to complementary probes or the like.

Detection can be carried out using hardware that resolves individual addresses in an array set forth herein. As such, a detection technique used in a method set forth herein can resolve any number of addresses up to an including all addresses in an array set forth herein. In some cases, optical labels can be detected using an optical detector. For example, luminophores (e.g. fluorophores) can be detected using a luminescence detector (e.g. fluorometer). A particularly useful configuration for detecting an array is an epiluminescence configuration. However, other configurations such as total internal reflection (TIR) can also be used.

A method set forth herein can be configured for high-throughput screening of affinity reagents. For example, a collection of different affinity reagent species can be initially subjected to a cyclic assay that is configured to distinguish affinity reagent species that have an association rate that is faster than a particular threshold rate from affinity reagent species that have an association rate under the threshold rate. Similarly, a collection of affinity reagent species can initially be subjected to a cyclic assay that is configured to distinguish affinity reagent species that have a dissociation rate under a particular threshold rate from affinity reagent species that have a dissociation rate over the threshold rate. Affinity reagent species that are on a desired side of a threshold can then be subjected to an assay that provides a more accurate or specific kinetic rate. As such, a triage process can employ an affinity reagent characterization method set forth herein but using fewer cycles than a subsequent process that is used to determine a kinetic rate. For example, a triage process can subject affinity reagents to at most 3, 2 or 1 cycles of an assay set forth herein, and a subsequent assay can subject one or more of the affinity reagents to at least 1, 2, 3 or more cycles of an assay. The conditions can be the same for the triage assay and subsequent assay. However, the condition need not be the same and can differ between the assay used for the triage process and the assay used in the subsequent process.

A screening process can start with a relatively large set of affinity reagent species for evaluation. For example, at least 10, 25, 50, 100, 1×10³ or more affinity reagent species can be subjected to a triage process. The triage process can be used to select a subset of the affinity reagent species for further analysis in an affinity reagent characterization method set forth herein. For example, at most 10%, 25%, 50%, 75%, or 90% of the affinity reagent species that were evaluated using a triage process can be subjected to a subsequent affinity reagent characterization method.

In some configurations of an affinity reagent characterization method, binding interactions between affinity reagents and immobilized proteins can be evaluated based on detection of affinity reagents in a fluid that is removed from contact with the proteins. For example, an array of proteins can be contacted with a fluid containing affinity reagents under conditions that are known or suspected to be appropriate for binding of the affinity reagents to the proteins. The fluid can then be removed from the array. Optionally, the removed fluid can be evaluated for presence of unbound affinity reagents. The array can subsequently be washed with one or more fluids and the fluid(s) can be removed from the array and evaluated for presence of affinity reagents. Fluid(s) that is (are) removed from an array can be monitored over time to track dissociation of affinity reagents. A trend in the number of affinity reagents of a particular species that are released can be used to determine a dissociation rate for the proteins of the array with that particular species of affinity reagents.

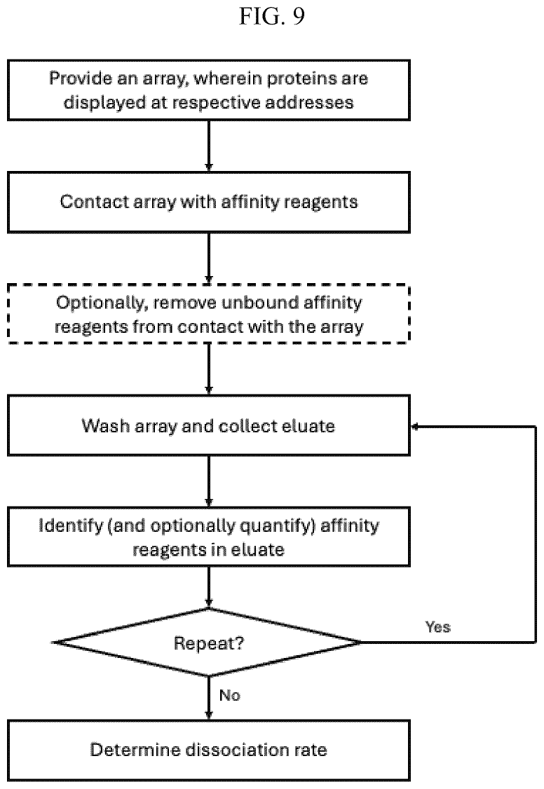

Accordingly, the present disclosure further provides a method of characterizing affinity reagents, including (a) providing an array, wherein the array includes a plurality of addresses, wherein a plurality of proteins is attached to the plurality of addresses, and wherein individual addresses of the array are each attached to a single protein of the plurality of proteins; (b) performing an assay, including (i) contacting the array with a set of affinity reagents, wherein the affinity reagents bind to proteins at addresses of the array, (ii) washing the array with a fluid, thereby collecting an eluate including affinity reagents that are dissociated from the array, (iii) detecting affinity reagents in the eluate, and (iv) repeating steps (ii) and (iii) for a plurality of cycles; and (c) determining a dissociation rate between the affinity reagents and the proteins based on the assay. Optionally, the affinity reagents include labels and the assay includes a step of detecting proteins at addresses of the array that are bound to affinity reagents of the set, wherein the detecting includes acquiring signals from the labels at respective addresses of the array, wherein the respective addresses are individually resolved. However, detection of affinity reagents on the array is not necessary. A diagrammatic representation of the method is shown in FIG. 9.

Optionally, affinity reagents include labels that uniquely identify the species of affinity reagent. For example, an affinity reagent can be attached to a nucleic acid that encodes the affinity reagent. A particularly convenient format for attaching an affinity reagent to its coding nucleic acid is a virus such as a phage in which an antibody or other proteinaceous affinity reagent is attached to a particle that contains the coding nucleic acid. In another example, an affinity reagent can be attached to a nucleic acid barcode that uniquely identifies the affinity reagent. For example, individual affinity reagents in a set of affinity reagents can be uniquely identifiable from other affinity reagents in the set via a respective barcode. Optionally, a nucleic acid barcode can be attached to an affinity reagent, for example via a retaining component such as a nucleic acid origami or other structured nucleic acid particle.

A nucleic acid can be detected and distinguished using any of a variety of nucleic acid sequence techniques. Particularly useful sequencing techniques are configured to separate nucleic acid species on arrays and sequence the nucleic acids in parallel. Nucleic acids can be sequenced, for example, using cyclical reversible terminator (CRT) sequencing technologies such as those that have been commercialized by Illumina, Inc. (e.g. HiSeq™, MiSeq™, NextSeq™, iSeq™ or NovaSeq™ platforms), BGI Genomics, or Singular Genomics (e.g. G4™ platform); sequencing by binding technologies such as those commercialized by Pacific Biosciences (e.g. Onso™ platform); sequencing by ligation technologies such as those commercialized by Life Technologies™ (e.g. ABI PRISM™, or SOLID™ platforms), real-time primer extension and detection sequencing techniques such as those commercialized by Pacific Biosciences (e.g. Revio™, Sequel™ or RS II™ systems), or nanopore sequencing techniques such as those commercialized by Oxford Nanopore (e.g. MinION™, GridION™ or PromethION™). Nucleic acids can also be detected using hybridization techniques such as those used to decode bead arrays (see, for example, U.S. Pat. No. 9,399,795, which is incorporated herein by reference) or using complementary primers in nucleic acid amplification techniques such as polymerase chain reaction or ligase chain reaction.

In some configurations, a method of characterizing affinity reagents can be used to evaluate a plurality of different affinity reagent species. The different affinity reagent species can be processed in parallel and distinguishably detected due to their attached nucleic acids. For example, a set of at least 2, 5, 10, 25, 50, 100, 500, 1×10³ or more different affinity reagent species can be contacted with an array as a pool. A set of different affinity reagent species can be collected from an array eluate and the nucleic acids sequenced, for example, as set forth above.

In addition to the foregoing reagents, also provided herein are kits useful in carrying out the analyses described herein, which kits may include the affinity reagents described above. The kits may optionally include one or more of enrichment reagents used to enrich for low abundance proteins and proteoforms, e.g., beads and antibodies used for the immune-isolation and/or immunoprecipitation of the proteins of interest, wash and other elution reagents, for such enrichment. Such kits may also include the flow-cells and arrays used to immobilize proteins of interest in a single molecule, in an optically detectable format for subsequent analysis in appropriately configured optical detection systems described herein. Such kits can include instructions for carrying out the enrichment, flow-cell deposition, interrogation and follow on analysis of biological samples using such kits.

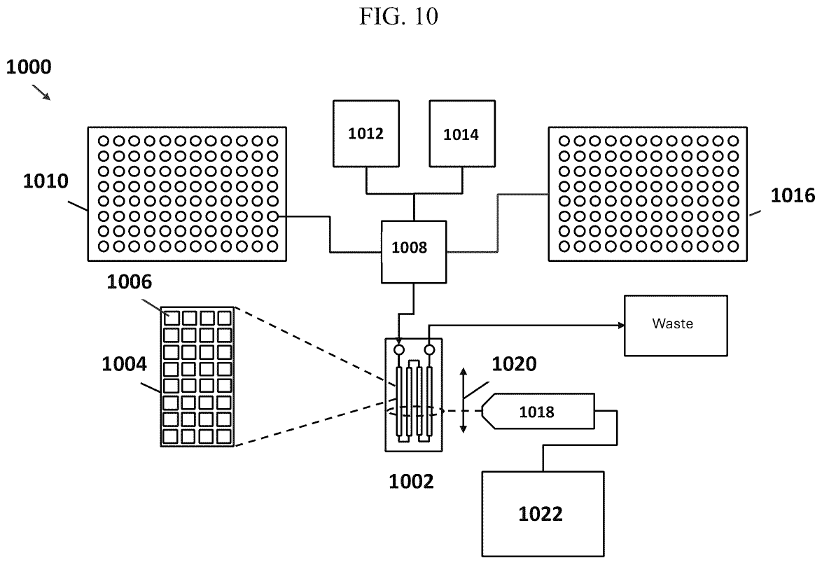

Additionally, provided herein are systems for performing the techniques, reagents, systems, and methods described herein. An example of a system is illustrated in FIG. 10. As shown, the system 1000 includes a flowcell 1002 that includes an array surface (shown as 1004) within the channels of the flow cell upon which individual protein molecules from a sample may be deposited and immobilized in locations 1006 that are individually addressable, and in particular cases are individually optically resolvable from each other using, e.g., fluorescence microscopy or scanning techniques.

The system will also typically include a fluidic delivery system 1008 that is configured to deliver different fluids to the flow cell 1002 through a series of fluidic lines and utilizing appropriate pumps, valves and other conventional fluid controls. The fluidics system 1008 may be fluidically coupled to various sources of fluids and reagents needed to carry out the analysis on the flow cell. For example, as shown, fluidic system 1008 is fluidly coupled to a source of a plurality of reagents 1010 (shown as a 96 well plate, although any number of different reagent storage systems of varying capacity may be employed) that includes a library of multiple affinity reagents that each have affinity for different characteristics of one or more proteins of interest. Additionally, fluidic system 1008 may also be coupled to sources of washing fluids or buffers 1012, and removal reagents 1014 (for removing bound affinity reagents following detection), as well as any other ancillary fluids and reagents needed for the analysis. Similarly, where flow cells are prepared on the system, the fluidic system may be coupled to sources of different sample materials that are to be analyzed 1016 (again, shown as a 96 well plate, although again, any suitable sample storage system or capacity may be suitable).

The reagents sources are typically fluidly connected to the flow-cell using fluidics systems that can separately access different reagents, sample materials and other fluids, and control the timing and volume of different reagents delivered to the flow-cell at different times in order to carry out the deposition, interrogation, washing and removal steps of the analysis process. Such fluidic systems will typically include requisite valves and pumps for carrying out such fluid deliveries and include, for example, those as described in, for example, International Patent Application No. WO 2023/122589A2, the full disclosure of which is hereby incorporated herein by reference in its entirety for all purposes.

The systems described herein also typically include a detection system, such as optical detection system 1018, for detecting and recording fluorescent signals arising from different positions on the array surface. Such detection systems may generally include line scanning confocal fluorescent microscope systems, which are capable of scanning across large array surfaces (as shown by arrow 1020) to detect and record fluorescence across such surfaces at reasonably high scan rates.

The overall systems also typically include one or more computers or processors 1022 for controlling the operation of the instrument system including the fluidic system 1008 (e.g., to sample different sample sources 1016, reagent sources 1010 and delivery timing and volume of each), and detection system 1018, among other functions, and for recording the detected signals received from the detection system 1018, e.g. fluorescent signals, and analyzing such signals to identify potential binding by each of the different affinity reagents. Processors 1022 also have access to memory storing instructions that are executed to perform any of the techniques described herein. Included in such memory may be bioinformatic software or firmware that evaluates the signals received and based upon appropriate modeling, identifies likely positive binding events, and then subsequently provides an overall assessment of characteristics of the proteins as described herein including identification information of proteins that are present at any given location on the array and/or the relative abundance of each different protein across the array and ultimately, within the sample being analyzed. Examples of bioinformatic software processes for analyzing such proteoform and proteome data have been described in, for example, U.S. Pat. Nos. 11,545,234, 10,473,654B1, and Egertson, et al., A theoretical framework for proteome-scale single-molecule protein identification using multi-affinity protein binding reagents, U.S. Patent Application No. 2022/0236282, International Patent Application Nos. PCT/US24/15132, and WO 2023/038859. Alternatively, in some cases, recorded data from the binding events, stored as digital information, digital image files, or compressed versions of such image files, may be transmitted to separate servers or cloud-based systems, which house the informatics software that performs this latter analysis and reporting.

The computer system 1022 can be an electronic device of a detection system, the electronic device being integral to the detection system or remotely located with respect to the detection system. The computer system 1022 includes a computer processing unit (CPU, also “processor” and “computer processor” herein), which can be a single core or multi core processor, or a plurality of processors for parallel processing. The computer system 1022 also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system 1022 can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. For example, one or more computer servers may enable cloud computing over the network (“the cloud”) to perform various aspects of analysis, calculation, and generation of the present disclosure, such as, for example, receiving information of empirical measurements of analytes in a sample; processing information of empirical measurements against a database comprising a plurality of candidate analytes, for example, using a binding model or function set forth herein; generating probabilities of a candidate analytes generating empirical measurements, and/or generating probabilities that extant analytes are correctly identified in the sample, and/or determining abundances of analytes in the sample. Such cloud computing may be provided by cloud computing platforms such as, for example, Amazon Web Services (AWS), Microsoft Azure, Google Cloud Platform, and IBM cloud. The network, in some cases with the aid of the computer system 1022, can implement a peer-to-peer network, which may enable devices coupled to the computer system 1022 to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system 1022 can be included in the circuit. In some cases, the circuit is an application specific integrated circuit (ASIC).

The storage unit can store files, such as drivers, libraries and saved programs. The storage unit can store user data, e.g., user preferences and user programs. The computer system 1022 in some cases can include one or more additional data storage units that are external to the computer system 1022, such as located on a remote server that is in communication with the computer system 1022 through an intranet or the Internet.

The computer system 1022 can communicate with one or more remote computer systems through the network. For instance, the computer system 1022 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1022 via the network.

Methods as described herein can be implemented by way of machine (e.g., computer processor) executable code stored on an electronic storage location of the computer system 1022, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1022, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1022 can include or be in communication with an electronic display that comprises a user interface (UI) for providing, for example, user selection of algorithms, binding measurement data, candidate proteins, and databases. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, receive information of empirical measurements of extant proteins in a sample, compare information of empirical measurements against a database comprising a plurality of protein sequences corresponding to candidate proteins, generate probabilities of a candidate protein generating the observed measurement outcome profile, and/or generate probabilities that candidate proteins are correctly identified in the sample, and/or generate abundances for the proteins in the sample.

The present disclosure provides a non-transitory information-recording medium that has, encoded thereon, instructions for the execution of one or more steps of the methods or techniques set forth herein, for example, when these instructions are executed by an electronic computer in a non-abstract manner. This disclosure further provides a computer processor (i.e. not a human mind) configured to implement, in a non-abstract manner, one or more of the methods set forth herein. All methods, compositions, devices and systems set forth herein will be understood to be implementable in physical, tangible and non-abstract form. The claims are intended to encompass physical, tangible and non-abstract subject matter. Explicit limitation of any claim to physical, tangible and non-abstract subject matter, will be understood to limit the claim to cover only non-abstract subject matter, when taken as a whole. Reference to “non-abstract” subject matter excludes and is distinct from “abstract” subject matter as interpreted by controlling precedent of the U.S. Supreme Court and the United States Court of Appeals for the Federal Circuit as of the priority date of this application.

### Example 1. Assay For Association Rate Determination

This example demonstrates an assay that can reliably assess the association rates for a variety of binding partner pairs including, but not limited to, proteins and affinity reagents that recognize the proteins. The assay set forth in this example was configured to monitor binding of a plurality of immobilized proteins to solution phase affinity reagents. The proteins were distributed to addresses of an array, whereby each protein was spatially separated from all other proteins in the array. The spatial separation allowed detection of binding between each protein and a respective affinity reagent to be resolved. Moreover, the array format allowed the results for multiple proteins to be detected and evaluated in parallel. As such, the format provided a multiplexed, single molecule-resolved binding assay. The assay can be extended to other analytes besides proteins and any of a variety of affinity reagents that interact with the analytes.

The assay was configured to use concentration titrations or time course titrations for various affinity reagents in order to reliably assess a proxy for association rates of those affinity reagents when binding to proteins immobilized at addresses of an array. The assay was demonstrated to determine association rates by varying affinity reagent concentration while incubation time was constant or by varying incubation time while affinity reagent concentration was constant.

Proteins were attached to structured nucleic acid molecules (SNAPs) to form molecular constructs including a single SNAP attached to a single protein (individual constructs being referred to as a “SNAP-P”). The SNAP-P constructs were formed and deposited on arrays as described in US Pat. App. Pub. No. 2023/0167488 A1 and U.S. Pat. No. 11,505,796, each of which is incorporated herein by reference. Affinity reagents were formed by attaching each of the SNAPs to 44 identical fluorescent labels and to 30 identical antibodies. As such, each of the affinity reagents included a SNAP attached to a plurality of labels and a plurality of antibodies (each of the affinity reagents being individually referred to as a “Lobe”). Antibodies were generated to recognize trimeric amino acid sequences in variable sequence contexts as described in U.S. patent application Ser. No. 18/448,000, which is incorporated herein by reference. Lobes were formed as described in U.S. Pat. No. 11,692,217, which is incorporated herein by reference.

The instrument for the assay included a custom fluorescence microscope configured to observe array surfaces within a multi-lane flow cell using optics configured for epifluorescent detection. The excitation wavelength was set at 647 nm and the emission wavelength was set at 671 nm. The instrument also included a fluidic system configured to deliver fluid reagents through an inlet of the flow cell to contact the surfaces of the array and then through an outlet of the flow cell to a waste receptacle. The instrument components are described in US Pat. App. Pub. No. 2023/0287480 A1 and PCT Publication No. WO 2023/122589, each of which is incorporated herein by reference.

Lobes were delivered to the SNAP-P arrays and binding was detected in iterative cycles. Each cycle included a Lobe delivery step, after an incubation period unbound Lobes were removed via a wash step, then Lobes bound to array addresses were detected, and finally Lobes were removed from the array in preparation for the next cycle. Lobe were delivered to the flow cell in Binding Solution (8.8 mM HEPES pH7.4, 130 mM NaCl, 4.4 mM KCl, 10.625 mM MgCl², 0.125 mM EDTA, 0.625 mM Tris pH 8.0, 0.1% Tween-20, 0.1% BSA, 1% Pluronic F127, 1 mg/ml Sheared Salmon DNA, 0.1% Proclin 150 (contains 1.55 mM Mg(NO₃)₂)). The flow cell was washed with Running Solution (50 mM HEPES pH7.4, 120 mM NaCl, 5 mM KCl, 10 mM MgCl₂, 0.1% Tween-20, 0.5% Procilin 150, 10 mM Sodium Sulfite, 10 mM Sodium L_Ascorbate) and detection was carried out in the Running Solution. Lobes were removed from the array by washing the flow cell with Remove Solution (100 mM CHAPs). The conditions were selected to retain SNAP-Ps on the array across multiple cycles. For determining concentration dependent association rates, different Lobe concentrations were delivered to the array across the cycles and incubated for a steady incubation period prior to detection. Up to 150 concentrations could be conveniently tested per lane. For determining time dependent association rates, a constant concentration of Lobes was delivered to the array across the cycles and incubated for various incubation periods prior to detection. Up to 28 time-points could be conveniently tested per lane.

Raw data was obtained from the instrument and processed as follows. For each cycle, remove failure from the previous cycle was subtracted from the current cycle to account for signal carryover from Lobes that failed to be removed from the array addresses. The resulting data was then plotted using Prism (GraphPad, Boston MA) in the Association Kinetics analysis mode.

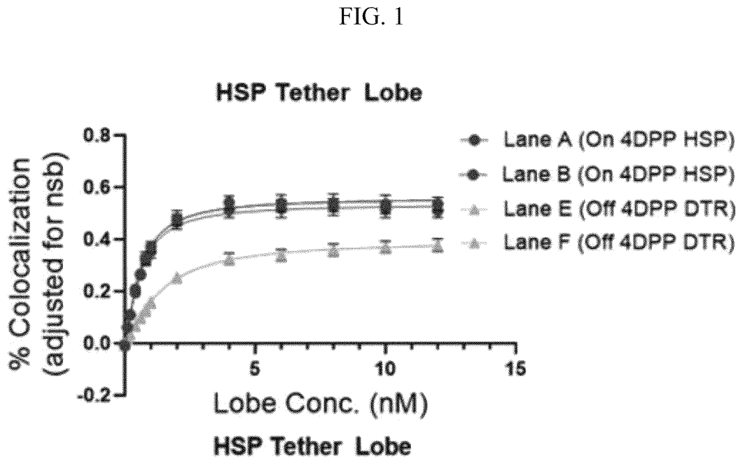

FIG. 1 shows a plot of HSP Lobe concentration vs. the percent of array addresses where Lobe was detected to be colocalized with a SNAP-P. The percent colocalization was adjusted for non-specific binding (NSB). Results from four different lanes of the flow cell, including lanes A and B, which contained arrays in which the SNAP-Ps were loaded with 4DPP peptides having HSP epitopes, and lanes E and F which contained arrays in which the SNAP-Ps were loaded with 4DPP peptides having DTR epitopes.

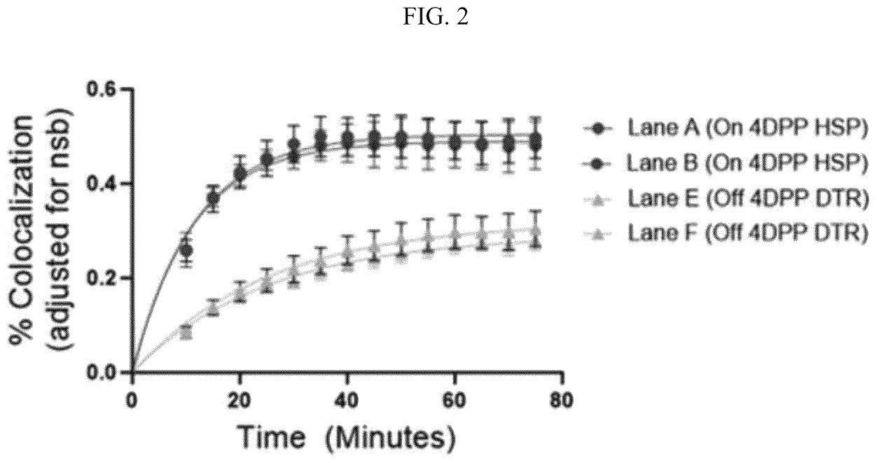

FIG. 2 shows a plot of time vs. the percent of array addresses where Lobe was detected to be colocalized with a SNAP-P. The percent colocalization was adjusted for non-specific binding (NSB). Results from four different lanes of the flow cell including lanes A and B which contained arrays in which the SNAP-Ps were loaded with 4DPP peptides having HSP epitopes, and lanes E and F which contained arrays in which the SNAP-Ps were loaded with 4DPP peptides having DTR epitopes.

The HSP Lobe had been previously determined to have relatively high affinity for the HSP epitope and low affinity for the DTR epitope. The results of FIGS. 1 and 2 were consistent with this observation and provided proxy association rates as well. The proxy association rates were 5.7×10⁵ M⁻¹ min⁻¹ for lane A, 4.2×10⁵ M⁻¹ min⁻¹ for lane B, 0.5×10³ M⁻¹ min⁻¹ for lane E, and 0.5×10³ M⁻¹ min⁻¹ for lane F.

### Example 2. Assay For Dissociation Rate Determination

This example demonstrates an assay that can reliably assess the dissociation rates for a variety of binding partner pairs including, but not limited to, proteins and affinity reagents that recognize them. The assay set forth in this example was configured to monitor dissociation of complexes, wherein each of the complexes includes an immobilized protein reversibly bound to an affinity reagent. The complexes were formed by (i) distributing proteins to addresses of an array, whereby individual proteins were immobilized at a respective address and spatially separated from all other immobilized proteins in the array; and (2) then incubating the array with a solution containing detectably labeled affinity reagents under conditions that allowed labeled affinity reagents to bind to immobilized proteins. The spatial separation allowed detection of protein-affinity reagent complexes at each address such that loss of signal indicated dissociation of the labeled affinity reagent from the addresses. Moreover, the array format allowed the results for multiple complexes to be detected and evaluated in parallel. As such, the format provided a multiplexed, single molecule-resolved dissociation assay. The assay can be extended to other analytes besides proteins and any of a variety of affinity reagents that interact with the analytes.

Protein arrays were formed using SNAP-Ps, the arrays were incubated with Lobes, and detected using an instrument as set forth in Example I, above. SNAP-Ps were contacted with array substrates at 150 pM and incubated for 30 minutes. After removing excess SNAP-Ps from contact with the arrays, the arrays were incubated with Lobes at a concentration of 10 nM for 30 minutes. A brief wash was then performed followed by a series of imaging cycles at regular intervals. Time was the only factor that changed over the cycles of imaging as no wash or reagent changes were performed between cycles. The resulting data was then plotted using Prism (GraphPad, Boston MA) in the Dissociation kinetics analysis mode.

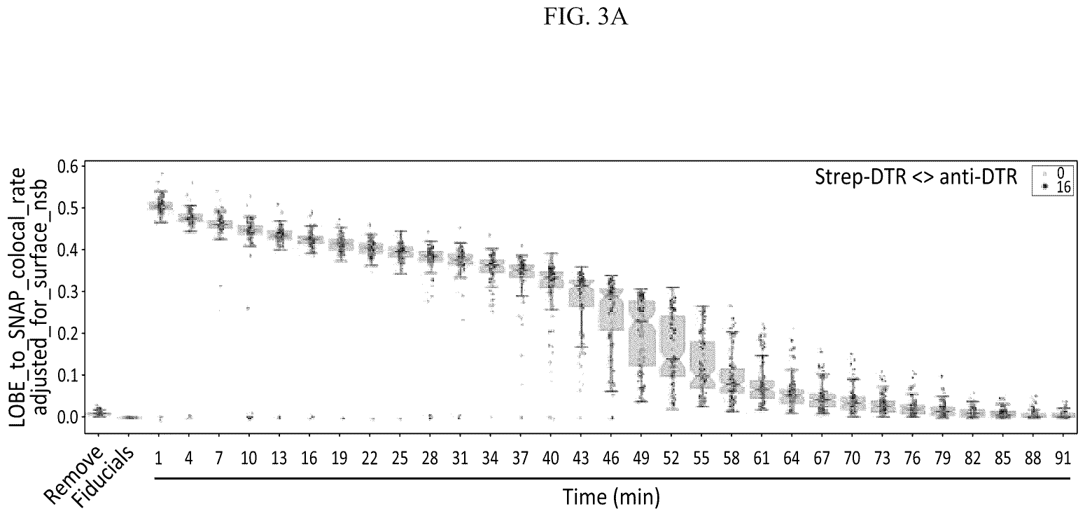

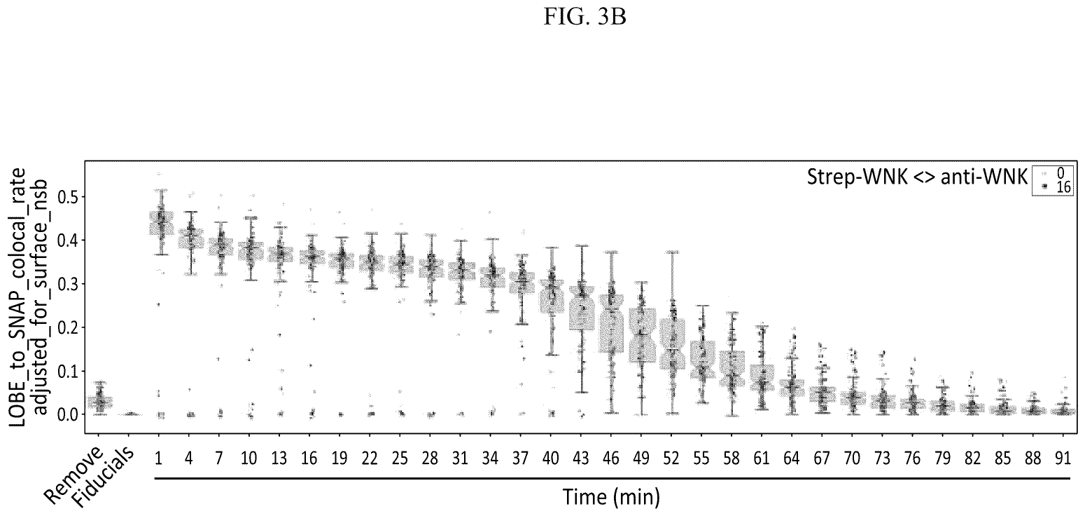

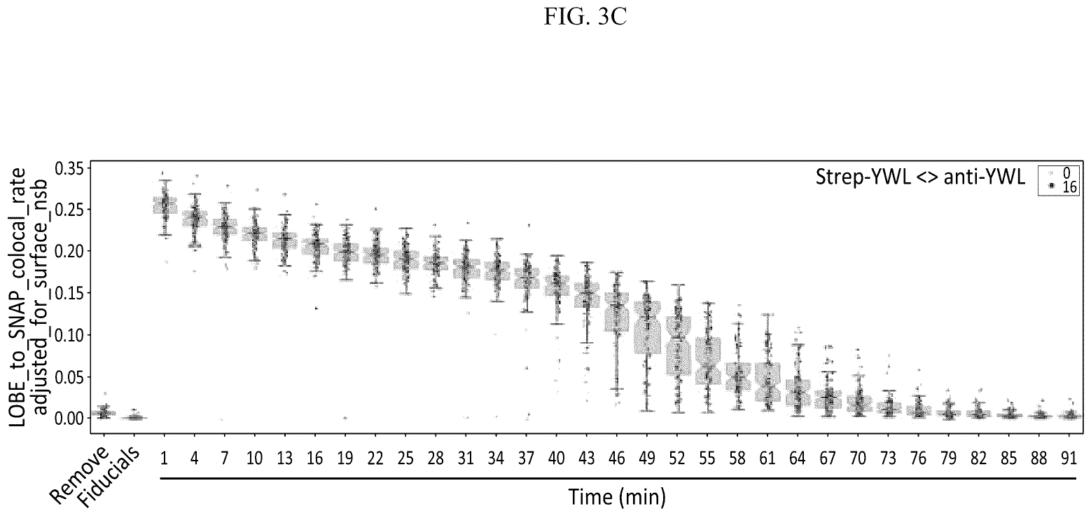

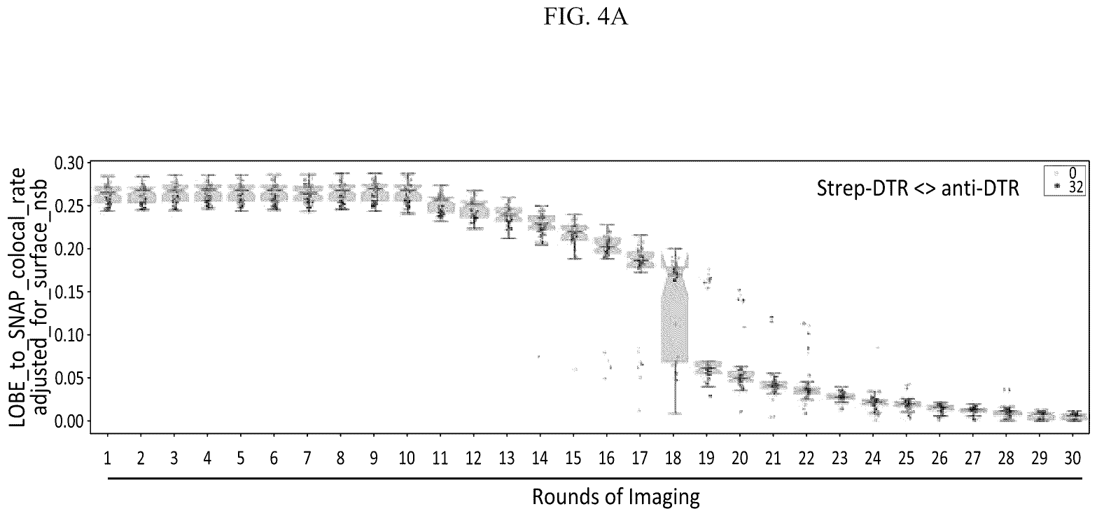

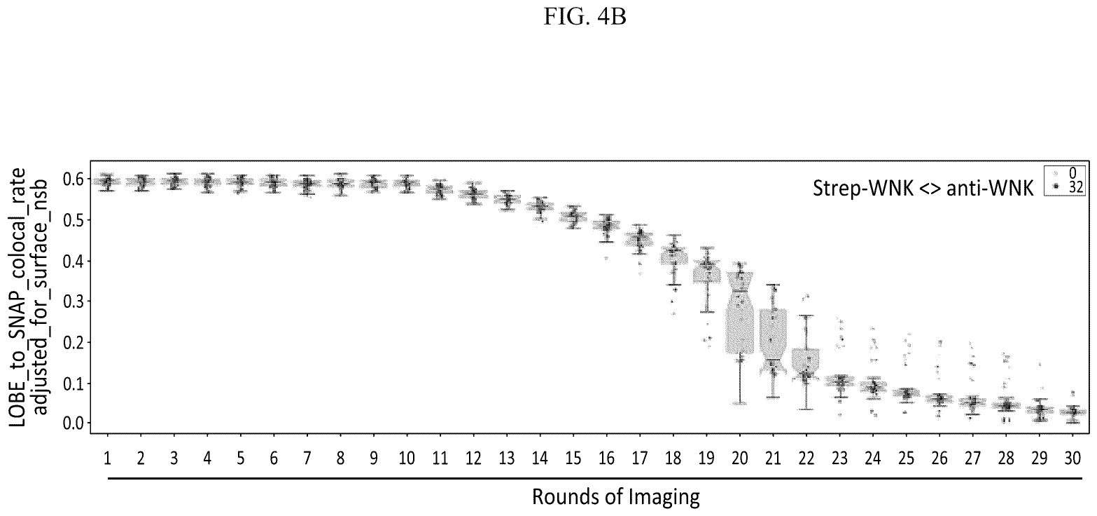

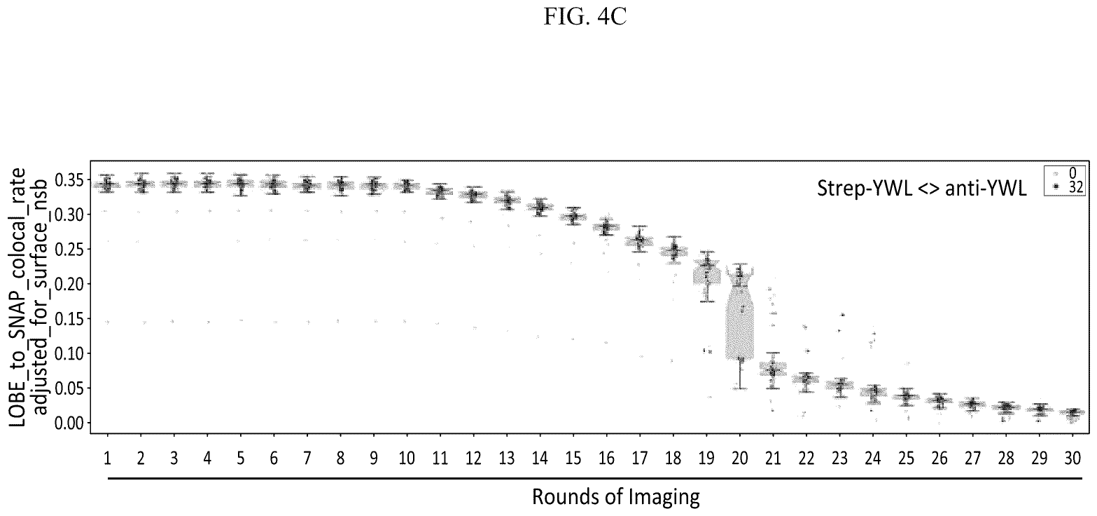

Initially, a photobleaching analysis was performed by periodically irradiating arrays containing complexes that had been formed between Lobes and SNAP-Ps loaded with peptides containing 3-mer amino acid sequence epitopes. Three different pairs of Lobes and peptides were tested including anti-DTR Lobes and peptides having the DTR amino acid sequence (see FIG. 3A), anti-WNK Lobes and peptides having the WNK amino acid sequence (see FIG. 3B), and anti-YWL Lobes and peptides having the YWL amino acid sequence (see FIG. 3C). Each pair was monitored in a separate flow cell lane from all other pairs. Photobleaching was observed over 30 cycles of excitation and emission detection. Decay was relatively slow for the first 15 cycles after which a substantial acceleration of decay was observed. The experiments were then repeated in the presence of 10 mM ascorbate (ascorbate was hypothesized to act as a photoprotectant and antioxidant). The results of repeated cycles of excitation and emission detection in the presence of ascorbate are shown in FIG. 4A for anti-DTR Lobes and peptides having the DTR amino acid sequence, FIG. 4B for anti-WNK Lobes and peptides having the WNK amino acid sequence, and FIG. 4C for anti-YWL Lobes and peptides having the YWL amino acid sequence. This data shows that the addition of ascorbate inhibited photobleaching for the first 10 cycles of imaging. It was concluded that monitoring dissociation of complexes over ten imaging cycles is sufficiently reliable to deduce dissociation rates.

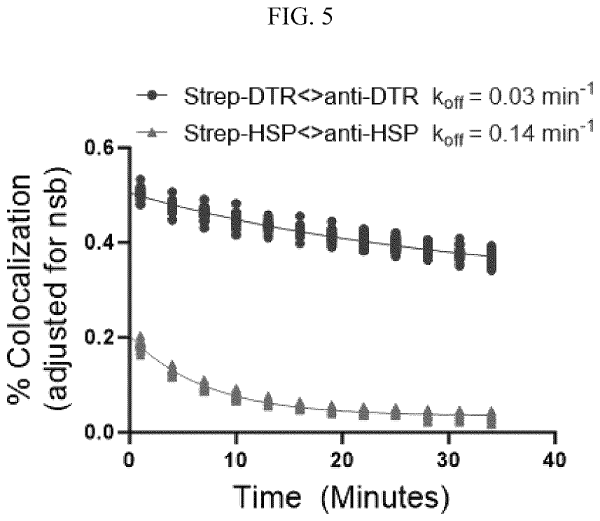

FIG. 5 shows a plot of percent colocalization of Lobes with array addresses over 35 minutes. The upper curve shows dissociation of anti-DTR Lobe from addresses that were previously attached to SNAP-Ps loaded with peptides having DTR amino acid sequences. The lower curve shows dissociation of anti-HSP Lobe from addresses that were previously attached to peptides having HSP amino acid sequences. The percent colocalization was adjusted for non-specific binding (NSB). The results showed that DTR Lobes bound to peptides having DTR epitopes with stronger binding and higher avidity compared to HSP Lobes bound to peptides having HSP epitopes. For DTR Lobes bound to DTR peptides the proxy k_{off} was 0.03 min⁻¹. For HSP Lobes bound to HSP peptides, the proxy k_{off} was 0.03 min⁻¹. The data clearly showed that HSP Lobes have a much faster dissociation rate compared to DTR Lobes, when bound to their respective epitopes.

### Example 3. Assay For Association Rate and Dissociation Rate Determinations

This example describes an assay configuration that combines the association rate kinetic assay of Example I with the dissociation rate kinetic assay of Example II. By combining the assays, this configuration provides for more rapid and cost-efficient determination of equilibrium constants such as an equilibrium dissociation constant (K_{D}) or equilibrium association constant (K_{A}). Furthermore, by performing both the association rate and dissociation rate measurement this configuration can provide for additional statistical analysis and, in some cases, a more accurate determination of kinetic rates for a given binding pair.

In this configuration the association rate kinetic assay was performed in the mode set forth in Example I for determining time dependent rates. After acquiring data for the last time point, the resulting array was subjected to the dissociation rate kinetic assay as set forth in Example II. The data was plotted using Prism (GraphPad, Boston MA) in the Association Then Dissociation kinetic analysis mode.

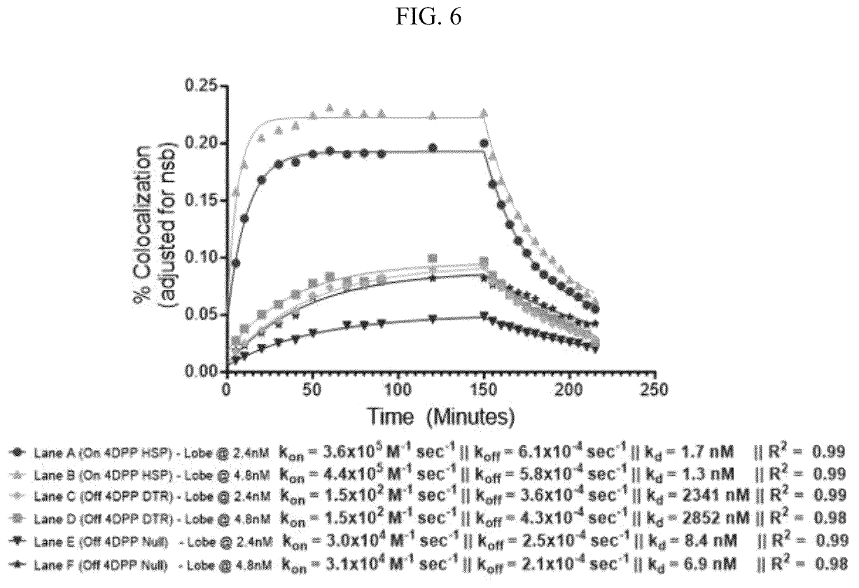

FIG. 6 shows a plot of percent colocalization of Lobes with array addresses over 230 minutes. The curves include replicate lanes analyzed for association and dissociation between anti-HSP Lobe and addresses containing SNAP-Ps loaded with peptides having HSP amino acid sequences. Also shown are curves for replicate lanes analyzed for association and dissociation between anti-DTR Lobe and addresses containing SNAP-Ps loaded with peptides having DTR amino acid sequences. Finally, curves are also shown for replicate lanes analyzed for association and dissociation between anti-HSP Lobe and addresses that were immobilized to SNAP-Ps that were not attached to any peptides (null). The percent colocalization was adjusted for non-specific binding (NSB) in all cases. The results indicated that association rates and dissociation rates can be obtained from the same flow cell on the instrument. For the HSP lanes, Lobe binding to HSP peptides had proxy kₒₙ values of 3.6×10⁵ M⁻¹ sec⁻¹ and 4.4×10⁵ M⁻¹ sec⁻¹, respectively; Lobe dissociation from HSP peptides had proxy k_{off} values of 6.1×10⁻⁴ sec⁻¹ and 5.8×10⁻⁴ sec⁻¹, respectively; the calculated proxy K_{D} values were 1.7 nM and 1.3 nM, respectively; and the R² for both fitted curves was 0.99. For the DTR lanes, Lobe binding to DTR peptides had proxy kₒₙ values of 1.5×10² M⁻¹ sec⁻¹ and 1.5×10² M⁻¹ sec⁻¹, respectively; Lobe dissociation from DTR peptides had proxy k_{off} values of 3.6×10⁻⁴ sec⁻¹ and 4.3×10⁻⁴ sec⁻¹, respectively; the calculated proxy K_{D} values were 2341 nM and 2852 nM, respectively; and the R² for the fitted curves were 0.99 and 0.98, respectively. For the null lanes, Lobe binding to null SNAPs had proxy kₒₙ values of 3.0×10⁴ M⁻¹ sec⁻¹ and 3.1×10⁴ M⁻¹ sec⁻¹, respectively; Lobe dissociation from null SNAPs had proxy k_{off} values of 2.5×10⁻⁴ sec⁻¹ and 2.1×10⁻⁴ sec⁻¹, respectively; the calculated proxy K_{D} values were 8.4 nM and 6.9 nM, respectively; and the R² for the fitted curves were 0.99 and 0.98, respectively. Proxy K_{D} values were calculated by dividing the proxy kₒₙ values by the proxy k_{off} values.

### Example 4

### Equilibrium Assay For Kinetic Parameter Determinations

This example describes an assay configuration that facilitates determination of association rates and dissociation rates simultaneously via single molecule measurements at equilibrium. The binding kinetics measurements can be made via a single contact of affinity reagents to a plurality of binding targets, although further measurements at the same affinity reagent concentration or differing affinity reagent concentration can be utilized to improve statistical measures.

An array of binding targets is provided. The array contains a glass solid support with a patterned layer of aluminum disposed on a surface of the glass. The aluminum layer is patterned with a plurality of nanowells spaced an optically-resolvable distance apart. Each array contains about 10¹⁰ nanowells. The bottom of each nanowell exposes a portion of the glass surface, and the glass surface is covered in a layer of oligonucleotides. The array is configured such that light can be passed through the glass, thereby illuminating the bottom region of the nanowell adjacent to the glass surface. The layer of oligonucleotides in each nanowell binds a single nucleic acid nanoparticle. The nucleic acid nanoparticle in each well is attached to a single binding target. Each binding target comprises an amino acid sequence DTR. The binding targets vary with respect to flanking sequences surrounding the amino acid sequence DTR. The flanking sequences each contain 0 to 2 amino acids, with the amino acids of the flanking sequences varied amongst all of the naturally-occurring amino acids (e.g., DTRA, DTRC, DTRD, . . . . ADTRA, ADTRC, ADTRD, ADTRE . . . CDTRA, CDTRC, CDTRD, . . . . YDTR, DTRAA, DTRAC, . . . . AADTRAA, etc.), thereby providing a library of 177221 unique binding targets. Each binding target is provided in a substantially equimolar amount, thereby providing about 56000 copies of each binding target distributed amongst the 10¹⁰ nanowells.

A fluidic medium comprising a plurality of DTR-specific antibodies is contacted to the array. The concentration of DTR-specific antibodies in the fluidic medium is 200 nanomolar (nM) before contacting the fluidic medium to the array of binding targets. Each antibody is attached to multiple Alexa-Fluor 647 fluorescent dyes. The fluidic medium is incubated with the array for 30 minutes to establish a binding equilibrium between the antibodies and the binding targets. After establishing equilibrium between the antibodies and binding targets, the array is imaged by fluorescence microscopy at an initial timepoint (t=0 seconds(s)). A series of images covering the entire array is collected at the initial timepoint, with each image collected capturing a region of the array containing about 10⁶ nanowells. Imaging is continued, with a complete series of images of the array captured once per second (i.e., a measurement frequency of 1 hertz (Hz)). Imaging occurs at the frequency of 1 Hz for 10 minutes, thereby providing 601 timepoints of data. Imaging data is transmitted to an image analysis algorithm on a computer processor.

The image analysis algorithm registers each array image for each measured timepoint. After registering the image, a presence or absence of a bound antibody is determined for each nanowell of the array by a detected or non-detected fluorescent signal at the nanowell address. Data across the 601 timepoints is compiled for each nanowell. For each nanowell, the length of time that the binding target in the nanowell is in a bound state and the length of time that the binding target is in an unbound state are calculated. Time lengths in the bound and unbound states are aggregated for each of the 177221 binding targets to provide an average length of time that the binding target is in a bound state, <t_{b, target}>, and an average length of time that the binding target is in an unbound state, <t_{u, target}>.

Kinetic rate parameters are calculated for each of the binding targets based upon the measured single-molecule equilibrium data. The second-order association rate constant, K_{on,target}, can be calculated as:

$${k}_{on , target} = \frac{1}{〈 {t}_{u , target} 〉 [ Antibody ]} \qquad ( 1 )$$

where [Antibody] is the initial concentration of antibody contacted to the array. The first-order dissociation rate constant, k_{off}, can be calculated as:

$${k}_{off , target} = \frac{1}{〈 {t}_{b , target} 〉} \qquad ( 2 )$$

The dissociation constant of the antibody to any particular binding target, K_{D,target}, can be calculated as:

$${K}_{D , target} = \frac{{k}_{off , target}}{{k}_{on , target}} \qquad ( 3 )$$

The experiment can be repeated at differing concentrations of antibody (e.g., about 100 nM, 300 nM, 400 nM, etc.). The fraction of any particular binding target bound by the antibodies, B_{target}, can be expected to depend upon the initial concentration of the antibody according to the equation:

$${B}_{t a rget} = \frac{x}{x + 1} \qquad ( 4 )$$

$$where x = [ Antibody ] / {K}_{D , target} .$$

Notwithstanding the appended claims, the disclosure set forth herein is also defined by the following clauses:
1. A method of characterizing affinity reagents, comprising
(a) providing an array, wherein the array comprises a plurality of addresses, wherein a plurality of proteins is attached to the plurality of addresses, and wherein individual addresses of the array are each attached to a single protein of the plurality of proteins;
(b) performing an assay, comprising
   (i) contacting the array with a set of affinity reagents, wherein the affinity reagents comprise optical labels,
   (ii) detecting binding of the affinity reagents to proteins at addresses of the array, wherein the detecting comprises acquiring optical signals from the optical labels at respective addresses of the array, wherein the respective addresses are individually resolved, and
   (iii) removing affinity reagents from the array,
      wherein steps (i) through (iii) are repeated for a plurality of cycles, each of the cycles using another set of affinity reagents instead of the set of affinity reagents, wherein affinity reagent species composition of the set of affinity reagents is identical to the other set of affinity reagents; and
(c) determining an association rate between the affinity reagents and the proteins based on the assay.
   2. The method of clause 1, wherein the plurality of addresses comprises a pitch of at least 250 nm.
   3. The method of clause 1 or 2, wherein the plurality of addresses comprises a pitch of at most 2 microns.
   4. The method of any one of the preceding clauses, wherein the plurality of addresses comprises at least 1000 addresses.
   5. The method of clause 4, wherein at least 500 of the addresses are each attached to a single respective protein of the plurality of proteins.
   6. The method of any one of the preceding clauses, wherein the detecting comprises resolving 500 or more addresses of the array.
   7. The method of any one of the preceding clauses, wherein individual addresses of the array are each attached to a single protein of the plurality of proteins via a structured nucleic acid particle.
   8. The method of clause 7, wherein the structured nucleic acid particle comprises a nucleic acid origami structure.
   9. The method of any one of the preceding clauses, wherein the affinity reagents comprise antibodies.
   10. The method of any one of clauses 1 through 7, wherein the affinity reagents comprise nucleic acid aptamers.
   11. The method of any one of the preceding clauses, wherein individual affinity reagents in the set of affinity reagents each comprise a retaining component.
   12. The method of clause 11, wherein the retaining component comprises a structured nucleic acid particle.
   13. The method of clause 12, wherein the structured nucleic acid particle comprises a nucleic acid origami structure.
   14. The method of any one of clauses 11 through 13, wherein the retaining component is attached to a plurality of luminescent labels.
   15. The method of clause 14, wherein the plurality of luminescent labels comprises at least 5 luminescent labels.
   16. The method of any one of clauses 11 through 15, wherein the retaining component is attached to a plurality of paratopes.
   17. The method of clause 16, wherein the plurality of paratopes comprises at least 5 paratopes.
   18. The method of any one of the preceding clauses, wherein the assay further comprises a wash step to remove unbound affinity reagents of the set from contact with the array after step (i) and before step (ii).
   19. The method of clause 18, further comprising performing a repeat of the assay, wherein conditions differ for the wash step of the assay compared to the repeat of the assay.
   20. The method of clause 19, wherein the conditions differ with respect to chemical composition of a wash solution used in the assay compared to chemical composition of a wash solution used in the repeat of the assay.
   21. The method of clause 19, wherein the conditions differ with respect to duration of the wash step in the assay compared to duration of the wash step in the repeat of the assay.
   22. The method of clause 19, wherein the conditions differ with respect to temperature of the array during the wash step in the assay compared to temperature of the array during the wash step in the repeat of the assay.
   23. The method of any one of the preceding clauses, wherein the optical labels comprise luminescent labels and the optical signals comprise luminescence.
   24. The method of clause 23, wherein individual affinity reagents in the set of affinity reagents are each attached to a plurality of luminescent labels.
   25. The method of clause 24, wherein the plurality of luminescent labels comprises at least 5 luminescent labels.
   26. The method of any one of clauses 23 through 25, wherein the luminescence is detected via epiluminescence microscopy.
   27. The method of any one of clauses 25 through 26, further comprising performing a repeat of the assay, wherein the number of luminescent labels in the plurality of luminescent labels differ for the assay compared to the repeat of the assay.
   28. The method of any one of the preceding clauses, wherein individual affinity reagents in the set of affinity reagents each comprise a plurality of paratopes.
   29. The method of clause 28, wherein the plurality of paratopes comprises at least 5 paratopes.
   30. The method of clause 28, further comprising performing a repeat of the assay, wherein the number of paratopes in the plurality of paratopes differ for the assay compared to the repeat of the assay.
   31. The method of any one of the preceding clauses, wherein the plurality of proteins consists essentially of proteins having identical amino acid sequences.
   32. The method of clause 31, wherein the plurality of proteins comprises at least 100 proteins having the identical amino acid sequences.
   33. The method of any one of the preceding clauses, wherein the plurality of cycles comprises at least 3 cycles.
   34. The method of clause 33, wherein the concentration of the set of affinity reagents differs between said at least 3 cycles.
   35. The method of any one of the preceding clauses, wherein the proteins comprise denatured tertiary structures.
   36. The method of any one of the preceding clauses, wherein the proteins comprise native tertiary structures.
   37. The method of any one of the preceding clauses, wherein the association rate between the affinity reagents and the proteins is determined from a count of a subset of addresses in the array from which the optical signals are acquired.
   38. The method of clause 37, wherein optical signals are acquired from the respective addresses in multiplicate signal acquisitions during step (ii) and wherein the association rate between the affinity reagents and the proteins is determined from an average of the multiplicate signal acquisitions.
   39. The method of clause 37 or 38, further comprising determining a statistical deviation for the average of the multiplicate signal acquisitions.
   40. The method of clause 39, wherein the statistical deviation comprises a standard deviation, dispersion, coefficient of variation, probability distribution, or frequency distribution.
   41. The method of clause 37, wherein a second association rate between the affinity reagents and the proteins is determined from a count of a second subset of addresses in the array from which the optical signals are acquired, wherein the association rate differs substantially from the second association rate.
   42. The method of clause 41, further comprising identifying a first subset of the proteins in the plurality of proteins as differing from a second subset of the proteins in the plurality of proteins based on the association rate and the second association rate.
   43. The method of any one of clauses 1 through 36, further comprising identifying a first subset of the proteins in the plurality of proteins as differing from a second subset of the proteins in the plurality of proteins based on different association rates determined for the first subset of the proteins compared to the second subset of the proteins.
   44. The method of clause 43, wherein the first subset of the proteins differs from the second subset of the proteins due to presence of a post-translational modification that is absent in the second subset of proteins.
   45. The method of clause 43, wherein the first subset of the proteins differs from the second subset of the proteins due to locations of amino acids that are modified for attachment to the addresses in the array.
   46. The method of any one of the preceding clauses, wherein concentration of the set of affinity reagents differs from concentration of the other set of affinity reagents.
   47. The method of any one of the preceding clauses, wherein duration of contact between the array and the set of affinity reagents differs from the duration of contact between the array and the other set of affinity reagents.
   48. The method of any one of the preceding clauses, wherein temperature during contact between the array and the set of affinity reagents differs from temperature during contact between the array and the other set of affinity reagents.
   49. The method of clause 1, wherein the assay further comprises steps of:
(iv) contacting the array with a further set of affinity reagents, wherein affinity reagent species composition of the set of affinity reagents is identical to the further set of affinity reagents, and wherein affinity reagents of the further set comprise optical labels,
(v) detecting proteins at addresses of the array that are bound to affinity reagents of the further set, wherein the detecting comprises acquiring optical signals from the optical labels at respective addresses of the array, wherein the respective addresses are individually resolved, and
(vi) repeating step (v) for a plurality of cycles, thereby detecting a decay in optical signals at the respective addresses of the array; and
further comprising (d) determining a dissociation rate between the affinity reagents of the further set and the proteins based on the assay.
   50. The method of clause 49, further comprising performing a wash step to remove unbound affinity reagents of the set from contact with the array after step (iv) and before step (v).
   51. The method of clause 50, further comprising performing a repeat of the further steps, wherein conditions differ for the wash step of the further steps compared to the repeat of the further steps.
   52. The method of clause 51, wherein the conditions differ with respect to chemical composition of a wash solution used in the further steps compared to chemical composition of a wash solution used in the repeat of the further steps.
   53. The method of clause 52, wherein the conditions differ with respect to duration of the wash step in the further steps compared to duration of the wash step in the repeat of the further steps.
   54. The method of clause 52, wherein the conditions differ with respect to temperature of the array during the wash step in the further steps compared to temperature of the array during the wash step in the repeat of the further steps.
   55. A method of characterizing affinity reagents, comprising
(a) providing an array, wherein the array comprises a plurality of addresses, wherein a plurality of proteins is attached to the plurality of addresses, and wherein individual addresses of the array are each attached to a single protein of the plurality of proteins;
(b) performing an assay, comprising
   (i) contacting the array with a set of affinity reagents, wherein the affinity reagents comprise optical labels, and wherein the affinity reagents bind to proteins at addresses of the array,
   (ii) detecting proteins at addresses of the array that are bound to affinity reagents of the set, wherein the detecting comprises acquiring optical signals from the optical labels at respective addresses of the array, wherein the respective addresses are individually resolved, and
   (iii) repeating step (ii) for a plurality of cycles, thereby detecting a decay in optical signals at the respective addresses of the array; and
(c) determining a dissociation rate between the affinity reagents and the proteins based on the assay.
   56. The method of clause 55, comprising performing the assay for multiple sets of affinity reagents, wherein species of affinity reagents differ between the multiple sets of affinity reagents.
   57. The method of clause 56, further comprising performing a repeat of the assay using a subset of the multiple sets of affinity reagents.
   58. The method of clause 57, wherein the performing of the assay for the multiple sets of affinity reagents comprises the repeating of step (ii) for a first plurality of cycles, wherein the performing of the repeat of the assay using the subset of the multiple sets of affinity reagents comprises the repeating of step (ii) for a second plurality of cycles, and wherein the second plurality of cycles is greater than the first plurality of cycles.
   59. The method of clause 58, wherein the first plurality of cycles comprises at most 2 cycles.
   60. The method of clause 58, wherein the subset of the multiple sets of affinity reagents comprises affinity reagents that are retained at the addresses of the array for a duration above a threshold duration.
   61. The method of any one of clauses 55 through 60, wherein the plurality of addresses comprises a pitch of at least 250 nm.
   62. The method of any one of clauses 55 through 61, wherein the plurality of addresses comprises a pitch of at most 10 microns.
   63. The method of any one of clauses 55 through 62, wherein the plurality of addresses comprises at least 1000 addresses.
   64. The method of clause 63, wherein at least 500 of the addresses are each attached to a single respective protein of the plurality of proteins.
   65. The method of any one of clauses 55 through 64, wherein the detecting comprises resolving 500 or more addresses of the array.
   66. The method of any one of clauses 55 through 65, wherein individual addresses of the array are each attached to a single protein of the plurality of proteins via a structured nucleic acid particle.
   67. The method of clause 66, wherein the structured nucleic acid particle comprises a nucleic acid origami structure.
   68. The method of any one of clauses 55 through 67, wherein the affinity reagents comprise antibodies.
   69. The method of any one of clauses 55 through 67, wherein the affinity reagents comprise nucleic acid aptamers.
   70. The method of any one of clauses 55 through 69, wherein individual affinity reagents in the set of affinity reagents each comprise a retaining component.
   71. The method of clause 70, wherein the retaining component comprises a structured nucleic acid particle.
   72. The method of clause 71, wherein the structured nucleic acid particle comprises a nucleic acid origami structure.
   73. The method of any one of clauses 70 through 72, wherein the retaining component is attached to a plurality of luminescent labels.
   74. The method of clause 73, wherein the plurality of luminescent labels comprises at least 5 luminescent labels.
   75. The method of any one of clauses 70 through 74, wherein the retaining component is attached to a plurality of paratopes.
   76. The method of clause 75, wherein the plurality of paratopes comprises at least 5 paratopes.
   77. The method of any one of clauses 55 through 76, wherein the assay further comprises a wash step to remove unbound affinity reagents of the set from contact with the array after step (i) and before step (ii).
   78. The method of clause 77, further comprising performing a repeat of the assay, wherein conditions differ for the wash step of the assay compared to the repeat of the assay.
   79. The method of clause 78, wherein the conditions differ with respect to chemical composition of a wash solution used in the assay compared to chemical composition of a wash solution used in the repeat of the assay.
   80. The method of clause 78, wherein the conditions differ with respect to duration of the wash step in the assay compared to duration of the wash step in the repeat of the assay.
   81. The method of clause 78, wherein the conditions differ with respect to temperature of the array during the wash step in the assay compared to temperature of the array during the wash step in the repeat of the assay.
   82. The method of any one of clauses 55 through 81, wherein the optical labels comprise luminescent labels and the optical signals comprise luminescence.
   83. The method of clause 82, wherein individual affinity reagents in the set of affinity reagents are each attached to a plurality of luminescent labels.
   84. The method of clause 83, wherein the plurality of luminescent labels comprises at least 5 luminescent labels.
   85. The method of any one of clauses 82 through 84, wherein the luminescence is detected via epiluminescence microscopy.
   86. The method of any one of clauses 83 through 85, further comprising performing a repeat of the assay, wherein the number of luminescent labels in the plurality of luminescent labels differ for the assay compared to the repeat of the assay.
   87. The method of any one of clauses 55 through 86, wherein individual affinity reagents in the set of affinity reagents each comprise a plurality of paratopes.
   88. The method of clause 87, wherein the plurality of paratopes comprises at least 5 paratopes.
   89. The method of clause 87, further comprising performing a repeat of the assay, wherein the number of paratopes in the plurality of paratopes differ for the assay compared to the repeat of the assay.
   90. The method of any one of clauses 55 through 89, wherein the plurality of proteins consists essentially of proteins having identical amino acid sequences.
   91. The method of clause 90, wherein the plurality of proteins comprises at least 100 proteins having the identical amino acid sequences.
   92. The method of any one of clauses 55 through 91, wherein the plurality of cycles comprises at least 3 cycles.
   93. The method of any one of clauses 55 through 92, wherein the proteins comprise denatured tertiary structures.
   94. The method of any one of clauses 55 through 92, wherein the proteins comprise native tertiary structures.
   95. The method of any one of clauses 55 through 94, wherein the dissociation rate between the affinity reagents and the proteins is determined from a count of a subset of addresses in the array from which the optical signals are acquired.
   96. The method of clause 95, wherein optical signals are acquired from the respective addresses in multiplicate signal acquisitions during step (ii) and wherein the dissociation rate between the affinity reagents and the proteins is determined from an average of the multiplicate signal acquisitions.
   97. The method of clause 95 or 96, further comprising determining a statistical deviation for the average of the multiplicate signal acquisitions.
   98. The method of clause 97, wherein the statistical deviation comprises a standard deviation, dispersion, coefficient of variation, probability distribution, or frequency distribution.
   99. The method of clause 95, wherein a second dissociation rate between the affinity reagents and the proteins is determined from a count of a second subset of addresses in the array from which the optical signals are acquired, wherein the dissociation rate differs substantially from the second dissociation rate.
   100. The method of clause 99, further comprising identifying a first subset of the proteins in the plurality of proteins as differing from a second subset of the proteins in the plurality of proteins based on the dissociation rate and the second dissociation rate.
   101. The method of any one of clauses 55 through 100, further comprising identifying a first subset of the proteins in the plurality of proteins as differing from a second subset of the proteins in the plurality of proteins based on different dissociation rates determined for the first subset of the proteins compared to the second subset of the proteins.
   102. The method of clause 101, wherein the first subset of the proteins differs from the second subset of the proteins due to presence of a post-translational modification that is absent in the second subset of proteins.
   103. The method of clause 101, wherein the first subset of the proteins differs from the second subset of the proteins due to locations of amino acids that are modified for attachment to the addresses in the array.
   104. A method of characterizing affinity reagents, comprising
(a) providing an array, wherein the array comprises a plurality of addresses, wherein a plurality of proteins is attached to the plurality of addresses, and wherein individual addresses of the array are each attached to a single protein of the plurality of proteins;
(b) performing an assay, comprising
   (i) contacting the array with a set of affinity reagents, wherein the affinity reagents bind to proteins at addresses of the array,
   (ii) washing the array with a fluid, thereby collecting an eluate comprising affinity reagents that are dissociated from the array,
   (iii) detecting affinity reagents in the eluate, and
   (iv) repeating steps (ii) and (iii) for a plurality of cycles; and
(c) determining a dissociation rate between the affinity reagents and the proteins based on the assay.
   105. The method of clause 104, wherein step (ii) optionally comprises, prior to the washing, detecting proteins at addresses of the array that are bound to affinity reagents of the set, wherein the detecting comprises acquiring optical signals from the optical labels at respective addresses of the array, wherein the respective addresses are individually resolved.
   106. The method of clause 104 or 105, wherein the affinity reagents comprise virus particles, the virus particles comprising proteinaceous paratope moieties and nucleic acids encoding the proteinaceous paratope moieties.
   107. The method of clause 106, wherein the detecting of the affinity reagents in the eluate comprises sequencing the nucleic acids encoding the proteinaceous paratope moieties.
   108. The method of any one of clauses 104 through 107, wherein the set of affinity reagents comprises a plurality of different affinity reagent species.
   109. The method of clause 104, comprising performing the assay for multiple sets of affinity reagents, wherein species of affinity reagents differ between the multiple sets of affinity reagents.
   110. The method of clause 109, further comprising performing a repeat of the assay using a subset of the multiple sets of affinity reagents.
   111. The method of clause 110, wherein the performing of the assay for the multiple sets of affinity reagents comprises the repeating of steps (ii) and (iii) for a first plurality of cycles, wherein the performing of the repeat of the assay using the subset of the multiple sets of affinity reagents comprises the repeating of steps (ii) and (iii) for a second plurality of cycles, and wherein the second plurality of cycles is greater than the first plurality of cycles.
   112. The method of clause 111, wherein the first plurality of cycles comprises at most 2 cycles.
   113. The method of any one of clauses 104 through 112, wherein the plurality of addresses comprises a pitch of at least 250 nm.
   114. The method of any one of clauses 104 through 113, wherein the plurality of addresses comprises a pitch of at most 10 microns.
   115. The method of any one of clauses 104 through 114, wherein the plurality of addresses comprises at least 1000 addresses.
   116. The method of clause 115, wherein at least 500 of the addresses are each attached to a single respective protein of the plurality of proteins.
   117. The method of any one of clauses 104 through 116, wherein individual addresses of the array are each attached to a single protein of the plurality of proteins via a structured nucleic acid particle.
   118. The method of clause 117, wherein the structured nucleic acid particle comprises a nucleic acid origami structure.
   119. The method of any one of clauses 104 through 118, wherein the affinity reagents comprise antibodies or nucleic acid aptamers.
   120. The method of any one of clauses 104 through 119, wherein the affinity reagents comprise nucleic acid barcodes and wherein the nucleic acid barcodes uniquely identify individual affinity reagents in the set of affinity reagents.
   121. The method of clause 106, wherein the detecting of the affinity reagents in the eluate comprises sequencing the nucleic acid bar codes.
   122. The method of any one of clauses 104 through 121, wherein individual affinity reagents in the set of affinity reagents each comprise a retaining component.
   123. The method of clause 122, wherein the retaining component comprises a structured nucleic acid particle.
   124. The method of clause 122 or 123, wherein the retaining component is attached to a plurality of paratopes.
   125. The method of any one of clauses 104 through 124, wherein conditions differ for the wash step when repeating steps (ii) and (iii).
   126. The method of clause 125, wherein the conditions differ with respect to chemical composition of the fluid used in step (ii) of the assay compared to chemical composition of the fluid used in the repeat of step (ii).
   127. The method of clause 125, wherein the conditions differ with respect to volume of the fluid used in step (ii) of the assay compared to volume of the fluid used in the repeat of step (ii).
   128. The method of clause 127, wherein the conditions differ with respect to temperature of the fluid used in step (ii) of the assay compared to temperature of the fluid used in the repeat of step (ii).
   129. The method of any one of clauses 104 through 128, wherein individual affinity reagents in the set of affinity reagents each comprise a plurality of paratopes.
   130. The method of clause 129, wherein the number of paratopes in the plurality of paratopes differ for the assay compared to the repeat of the assay.
   131. The method of any one of clauses 104 through 130, wherein the plurality of proteins consists essentially of proteins having identical amino acid sequences.
   132. The method of clause 131, wherein the plurality of proteins comprises at least 100 proteins having the identical amino acid sequences.
   133. The method of any one of clauses 104 through 132, wherein the plurality of cycles comprises at least 3 cycles.
   134. The method of any one of clauses 104 through 133, wherein the proteins comprise denatured tertiary structures.
   135. The method of any one of clauses 104 through 133, wherein the proteins comprise native tertiary structures.
   136. A method of characterizing affinity reagents, comprising
(a) providing an array, wherein the array comprises a plurality of addresses, wherein a plurality of proteins is attached to the plurality of addresses, and wherein individual addresses of the array are each attached to a single protein of the plurality of proteins;
(b) performing an assay, comprising
   (i) contacting the array with a set of affinity reagents, wherein the affinity reagents comprise optical labels,
   (ii) removing unbound affinity reagents from contact with the array, and
   (iii) contacting the array with a second set of affinity reagents, wherein the affinity reagents of the second set lack the optical labels and wherein the affinity reagents of the second set are identical species to the affinity reagents of the set of affinity reagents, and
   (iv) detecting binding of the affinity reagents to proteins at addresses of the array, wherein the detecting comprises acquiring optical signals from the optical labels at respective addresses of the array, wherein the respective addresses are individually resolved; and
(c) determining equilibrium binding constant for binding of the affinity reagents to the proteins based on the assay.
   137. The method of clause 136, wherein step (b) further comprises, (v) repeating step (iv) for a plurality of cycles, thereby detecting a change in optical signals at the respective addresses of the array.
   138. The method of any one of clauses 136 through 137, wherein the plurality of addresses comprises a pitch of at least 250 nm.
   139. The method of any one of clauses 136 through 138, wherein the plurality of addresses comprises a pitch of at most 10 microns.
   140. The method of any one of clauses 136 through 139, wherein the plurality of addresses comprises at least 1000 addresses.
   141. The method of clause 140, wherein at least 500 of the addresses are each attached to a single respective protein of the plurality of proteins.
   142. The method of any one of clauses 136 through 141, wherein the detecting comprises resolving 500 or more addresses of the array.
   143. The method of any one of clauses 136 through 142, wherein individual addresses of the array are each attached to a single protein of the plurality of proteins via a structured nucleic acid particle.
   144. The method of clause 143, wherein the structured nucleic acid particle comprises a nucleic acid origami structure.
   145. The method of any one of clauses 136 through 144, wherein the affinity reagents comprise antibodies.
   146. The method of any one of clauses 136 through 144, wherein the affinity reagents comprise nucleic acid aptamers.
   147. The method of any one of clauses 136 through 146, wherein individual affinity reagents in the set of affinity reagents each comprise a retaining component.
   148. The method of clause 147, wherein the retaining component comprises a structured nucleic acid particle.
   149. The method of clause 148, wherein the structured nucleic acid particle comprises a nucleic acid origami structure.
   150. The method of any one of clauses 147 through 149, wherein the retaining component is attached to a plurality of luminescent labels.
   151. The method of clause 150, wherein the plurality of luminescent labels comprises at least 5 luminescent labels.
   152. The method of any one of clauses 147 through 151, wherein the retaining component is attached to a plurality of paratopes.
   153. The method of clause 152, wherein the plurality of paratopes comprises at least 5 paratopes.
   154. The method of any one of clauses 136 through 153, wherein the optical labels comprise luminescent labels and the optical signals comprise luminescence.
   155. The method of clause 154, wherein individual affinity reagents in the set of affinity reagents are each attached to a plurality of luminescent labels.
   156. The method of clause 155, wherein the plurality of luminescent labels comprises at least 5 luminescent labels.
   157. The method of any one of clauses 154 through 156, wherein the luminescence is detected via epiluminescence microscopy.
   158. The method of any one of clauses 136 through 157, wherein individual affinity reagents in the set of affinity reagents each comprise a plurality of paratopes.
   159. The method of clause 158, wherein the plurality of paratopes comprises at least 5 paratopes.
   160. The method of clause 158, further comprising performing a repeat of the assay, wherein the number of paratopes in the plurality of paratopes differ for the assay compared to the repeat of the assay.
   161. The method of any one of clauses 136 through 160, wherein the plurality of proteins consists essentially of proteins having identical amino acid sequences.
   162. The method of clause 161, wherein the plurality of proteins comprises at least 100 proteins having the identical amino acid sequences.
   163. The method of any one of clauses 136 through 162, wherein the proteins comprise denatured tertiary structures.
   164. The method of any one of clauses 136 through 162, wherein the proteins comprise native tertiary structures.
   165. A system for characterizing affinity reagents, comprising:
(a) a solid support comprising a plurality of binding targets, wherein the solid support comprises a plurality of addresses, wherein only one binding target of the plurality of binding targets is immobilized to each address of the plurality of addresses, and wherein each address is individually resolvable from each other address of the plurality of addresses;
(b) a fluidics system that is configured to deliver a fluid comprising a plurality of affinity reagents to the solid support, wherein each affinity reagent comprises a detectable label that is configured to produce optical signals;
(c) an optical detector, wherein the optical detector is configured to detect optical signals from detectable labels of affinity reagents at addresses of the plurality of addresses; and
(d) a processor, wherein the processor is configured to receive data comprising presence or absence of an optical signal at each address of the plurality of addresses at a first timepoint and a second timepoint, and wherein the processor is further configured to characterize binding kinetics of the affinity reagents for the binding targets based upon the received data for the first timepoint and the second timepoint.
   166. The system of clause 165, wherein the binding kinetics includes an association rate or dissociation rate of the affinity reagents for the binding targets.
   167. A computer program product, comprising one or more non-transitory computer-readable media having computer program instructions stored therein, the computer program instructions being configured such that, when executed by one or more computing devices, the computer program instructions cause the one or more computing devices to:
receive first quantity data indicating a first quantity of affinity reagents bound to binding targets of a plurality of binding targets at a first timepoint;
   receive second quantity data indicating a second quantity of affinity reagents bound to binding targets of a plurality of binding targets at a second timepoint, the first timepoint and the second timepoint being different;
   determine, based upon a difference between the first quantity and the second quantity, binding kinetics of the affinity reagents for the binding targets.
   168. The system of clause 167, wherein the binding kinetics includes an association rate or dissociation rate of the affinity reagents for the binding targets.

What is claimed is:

1. A method of characterizing an affinity reagent, comprising:
(a) contacting a plurality of affinity reagents to a plurality of binding targets;
(b) detecting at single-analyte resolution a first quantity of affinity reagents bound to binding targets of the plurality of binding targets at a first timepoint;
(c) detecting at single-analyte resolution a second quantity of affinity reagents bound to binding targets of the plurality of binding targets at a second timepoint; and
(d) based upon a difference between the first quantity and second quantity of affinity reagents bound to binding targets of the plurality of binding targets, determining an association rate of the affinity reagents for the binding targets.

2. The method of claim 1, wherein contacting the plurality of affinity reagents to the plurality of binding targets comprises contacting the plurality of affinity reagents to an array of binding targets.

3. The method of claim 2, wherein the array of binding targets comprises a solid support comprising a plurality of addresses, wherein only one binding target of the plurality of binding targets is immobilized at each address of the plurality of addresses.

4. The method of claim 3, wherein each address comprises a well, wherein the only one binding target is immobilized in the well.

5. The method of claim 2, wherein each address is optically resolvable from any other address of the plurality of addresses.

6. The method of claim 5, wherein the plurality of addresses has a pitch of at least 500 nanometers (nm).

7. The method of claim 1, wherein each individual affinity reagent of the plurality of affinity reagents is attached to a detectable label.

8. The method of claim 7, wherein the detectable label is configured to provide an optical signal.

9. The method of claim 7, wherein detecting at single-analyte resolution a first quantity of affinity reagents bound to binding targets of the plurality of binding targets comprises detecting optical signals from the detectable labels of the first quantity of affinity reagents bound to the binding targets of the plurality of binding targets.

10. The method of claim 1, further comprising detecting at single-analyte resolution quantities of affinity reagents bound to binding targets of the plurality of binding targets for at least five different timepoints.

11. The method of claim 10, wherein the association rate of the affinity reagents for the binding targets is determined based upon the differences in quantities of affinity reagents bound to binding targets of the plurality of binding targets for the at least five different timepoints.

12. The method of claim 1, further comprising removing the bound affinity reagents from the plurality of binding targets.

13. The method of claim 12, wherein removing the bound affinity reagents from the plurality of binding targets occurs between steps (b) and (c).

14. The method of claim 13, further comprising contacting a second plurality of affinity reagents to the plurality of binding targets.

15. The method of claim 14, wherein the plurality of affinity reagents is contacted to the plurality of binding targets for a first incubation time and wherein the second plurality of affinity reagents is contacted to the plurality of binding targets for a second incubation time, wherein the first incubation time differs from the second incubation time.

16. The method of claim 1, further comprising rinsing unbound affinity reagents from the plurality of binding targets.

17. The method of claim 16, wherein the rinsing of unbound affinity reagents from the plurality of binding targets occurs before step (b) or (c).

18. The method of claim 1, wherein step (b) or (c) occurs in the presence of unbound affinity reagents.

19. A method of characterizing an affinity reagent, comprising:
(a) contacting a plurality of affinity reagents to a plurality of binding targets;
(b) detecting at single-analyte resolution a first quantity of affinity reagents bound to binding targets of the plurality of binding targets at a first timepoint;
(c) detecting at single-analyte resolution a second quantity of affinity reagents bound to binding targets of the plurality of binding targets at a second timepoint; and
(d) based upon a difference between the first quantity and second quantity of affinity reagents bound to binding targets of the plurality of binding targets, determining a dissociation rate of the affinity reagents for the binding targets.

20. A system for characterizing affinity reagents, comprising:
(a) a solid support comprising a plurality of binding targets, wherein the solid support comprises a plurality of addresses, wherein only one binding target of the plurality of binding targets is immobilized to each address of the plurality of addresses, and wherein each address is individually resolvable from each other address of the plurality of addresses;
(b) a fluid comprising a plurality of affinity reagents, wherein each affinity reagent comprises a detectable label that is configured to produce optical signals;
(c) a fluidics system that is configured to deliver the fluid comprising the plurality of affinity reagents to the solid support;
(d) an optical detector, wherein the optical detector is configured to detect optical signals from detectable labels of affinity reagents at addresses of the plurality of addresses; and
(e) a processor, wherein the processor is configured to receive data comprising presence or absence of an optical signal at each address of the plurality of addresses at a first timepoint and a second timepoint, and wherein the processor is further configured to determine an association rate of the affinity reagents for the binding targets based upon the received data for the first timepoint and the second timepoint.
